(19) United States
(12) Patent Application Publication
(10) Pub. No.: US 2024/0261447 A1
(43) Pub. Date: Aug. 8, 2024

(54) COMPOSITIONS AND METHODS FOR TREATING CONDITIONS RELATED TO THE HEPATOPANCREATIC ANATOMICAL REGION

(71) Applicant: THE REGENTS OF THE UNIVERSITY OF MICHIGAN, Ann Arbor, MI (US)

(72) Inventors: James J. Moon, Ann Arbor, MI (US); Cheng XU, Ann Arbor, MI (US); Timothy FRANKEL, Ann Arbor, MI (US); Peter SCOTT, Ann Arbor, MI (US)

(21) Appl. No.: 18/293,217

(22) PCT Filed: Jul. 25, 2022
(86) PCT No.: PCT/US2022/038155

§ 371 Date: Jan. 29, 2024

Related U.S. Application Data
(60) Provisional application No. 63/227,538, filed on Jul. 30, 2021.

(51) Int. Cl. A61K 51/12; A61K 39/44; A61K 45/06; A61K 47/69; A61K 49/00; A61K 51/06; A61P 1/18

(52) U.S. Cl. CPC A61K 51/1244; A61K 39/44; A61K 45/06; A61K 47/6927; A61K 47/6929; A61K 47/6939; A61K 49/0091; A61K 49/0093; A61K 51/065; A61P 1/18

(57) ABSTRACT

The present invention relates to biodegradable agents associated with (e.g., complexed, conjugated, encapsulated, absorbed, adsorbed, admixed) one or more of a therapeutic agent (e.g., configured for treating, preventing or ameliorating various types of disorders), a diagnostic agent, and an agent capable of relaxing (e.g., opening, dilating) the hepatopancreatic anatomical region, as well as systems and methods utilizing such compositions (e.g., in diagnostic and/or therapeutic settings (e.g., treating pancreatic cancers)).

### CROSS-REFERENCE TO RELATED APPLICATION

The present application claims priority to U.S. Provisional Patent Application No. 63/227,538, filed Jul. 30, 2021, which is hereby incorporated by reference in its entirety.

### FIELD OF THE INVENTION

The present invention relates to biodegradable agents associated with (e.g., complexed, conjugated, encapsulated, absorbed, adsorbed, admixed) one or more of a therapeutic agent (e.g., configured for treating, preventing or ameliorating various types of disorders), a diagnostic agent, and an agent capable of relaxing (e.g., opening, dilating) the hepatopancreatic anatomical region, as well as systems and methods utilizing such compositions (e.g., in diagnostic and/or therapeutic settings (e.g., treating pancreatic cancers)).

### BACKGROUND OF THE INVENTION

Pancreatic cancer is one of the most lethal cancers. The mortality approaches 100% because of the propensity for early metastatic spread, and because the disease is highly resistant to radiation and chemotherapy. Given that 27,000 new cases are diagnosed every year in North America and 68,000 in Europe, there is an urgent need to develop novel treatment strategies to reduce the mortality of pancreatic cancer patients. Pancreatic ductal adenocarcinoma (PDAC) is one of the most malignant cancers, and numerous therapeutic approaches have been explored based on drug response findings from pancreatic cancer cells tested in culture models.

For the treatment of pancreatic cancer in the clinic, chemo-drug formulations are typically given via an intravenous route. This also includes the newly FDA-approved liposomal irinotecan. However, recent studies that analyzed data from over 200 reports (2005-2015) have shown that a small dose of chemo-drug formulations (<0.7%) given via systemic route reach tumor tissues (Nature reviews materials, 2016, 1, 1-12), thus leading to elevated systemic exposure and serious off-target toxicity. While oral formulations of chemo-drug have been reported, this approach is mainly used either for systemic absorption of drug molecules or for mucoadhesive binding of drug carriers onto the intestinal wall, which leads to minimal drug accumulation in the target hepatopancreatic area.

Accordingly, improved methods for treating pancreatic malignancies and PDAC are needed. In addition, improved strategies for targeting therapeutics to the pancreas and biliary tree are needed.

The present invention addresses these needs.

### SUMMARY

Recent studies have shown that commensal gut microbes can enter the pancreas through the hepatopancreatic anatomical region (e.g., hepatopancreatic ampulla, hepatopancreatic duct, Ampulla of Vater, Sphincter of Oddi (e.g., a small opening that connects the pancreatic duct to the duodenum)). Taking cues from this, experiments conducted during the course of developing embodiments for the present invention determined that micro-sized or nano-sized particles (MPs) given via oral route can directly enter the pancreas. When such microparticles or nanoparticles are co-administered with commercial drugs known to relax the hepatopancreatic anatomical region (e.g., hepatopancreatic ampulla, hepatopancreatic duct, Ampulla of Vater, Sphincter of Oddi), this led to even further improvement in the accumulation of microparticles or nanoparticles in the pancreas.

Based on murine studies described herein, it was envisioned that chemotherapeutic-loaded micro or nanoparticles of a certain size range (e.g., 0.1 to 100 μm in diameter) can effectively deliver chemotherapeutic agents to the pancreas after oral administration, while significantly decreasing the systemic exposure of chemo-drugs and reducing off-target toxicity. Once the chemo-drugs loaded microparticles or nanoparticles enter the pancreas, they diffuse throughout the whole pancreas and remain in pancreas for hours to days. In the meantime, the pay load can be slowly released within the pancreas, thus greatly improving the bioavailability and pharmacokinetics of chemo-drugs.

Accordingly, the present invention relates to biodegradable agents associated with (e.g., complexed, conjugated, encapsulated, absorbed, adsorbed, admixed) one or more of a therapeutic agent (e.g., configured for treating, preventing or ameliorating various types of disorders), a diagnostic agent, and an agent capable of relaxing (e.g., opening, dilating) the hepatopancreatic anatomical region, as well as systems and methods utilizing such compositions (e.g., in diagnostic and/or therapeutic settings (e.g., treating pancreatic cancers)).

In certain embodiments, the present invention provides compositions comprising a biodegradable agent (e.g., a microparticle or a nanoparticle) (e.g., particle having a diameter between approximately 0.1 to 100 μm) associated with one or more of a therapeutic agent, a diagnostic agent, and an agent capable of relaxing (e.g., opening, dilating) the hepatopancreatic anatomical region. For example, in some embodiments, the present invention provides compositions comprising biodegradable agents (e.g., microparticles or nanoparticles) associated with (e.g., complexed, conjugated, encapsulated, absorbed, adsorbed, admixed) one or more of diagnostic agents, an agent capable of relaxing (e.g., opening, dilating) the hepatopancreatic anatomical region, and a therapeutic agent (e.g., agents capable of treating and/or ameliorating the symptoms of conditions associated with the pancreas (e.g., pancreatic cancer, pancreatitis, diabetes), conditions associated with the bile duct (e.g., cancer associated with the bile duct, cholangitis), conditions associated with the liver (e.g., liver cancer, hepatitis), conditions associated with the gall bladder (e.g., gall bladder cancer, gall stones, cholecystitis).

In some embodiments, the size of the diameter of the biodegradable agent is between approximately 0.1 to 100 μm (e.g., 0.001 μm, 0.01 μm, 0.1 μm, 0.5 μm, 1 μm, 1.5 μm, 1.999 μm, 2 μm, 5 μm, 10 μm, 15 μm, 19 μm, 19.9999 μm, 20 μm, 20.01 μm, 20.5 μm, 21 μm, 25 μm, 30 μm, 50 μm, 70 μm, 80 μm, 100 μm, 101 μm, 105 μm, 115 μm) (e.g., 0.001 to 115 μm, 0.1 to 105 μm, 0.5 to 101 μm, 1 to 100 μm, 1.9999 to 80 μm, 2 to 70 μm, 5 to 50 μm, 10 to 30 μm, 15 to 25 μm, 19 to 21 μm, 19.9999 to 20.5 μm, 20 to 20.01 μm). In some embodiments, the size of the diameter of the biodegradable agent is between approximately 5 to 20 μm.

In some embodiments, biodegradable agent is a microparticle or nanoparticle.

In some embodiments, the size of the microparticle is between 0.1 microns to 100 microns.

In some embodiments, the microparticle is a polystyrene microparticle.

In some embodiments, the microparticle is a bovine serum albumin microparticle. In some embodiments, the microparticle is a human serum albumin microparticle. In some embodiments, the microparticle is a bovine serum albumin microparticle.

In some embodiments, the microparticle is a corn zein microparticle.

In some embodiments, the microparticle is a biocompatible polymer selected from the group consisting of poly (lactide), poly(lactide-co-glycolide)s, poly (caprolactone), polycarbonates, polyamides, polyanhydrides, polyamino acids, polyortho esters, polyacetals, polycyanoacrylates, degradable polyurethanes, polyacrylates, polymers of ethylene-vinyl acetate and other acyl substituted cellulose acetates and derivatives thereof, polysaccharide, non-erodible polyurethanes, polystyrenes, polyvinyl chloride, polyvinyl fluoride, poly (vinyl imidazole), chlorosulphonated polyolifins, polyethylene oxide, and copolymers and mixtures thereof.

In some embodiments, the microparticle can comprise polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polyureas, polystyrenes, and/or polyamines. In some embodiments, a polymeric matrix may comprise poly(lactic-co-glycolic acid) (PLGA), polyethylene glycol (PEG), and/or copolymers thereof. In some embodiments, a polymeric particle can comprise dendrimers, proteins, carbohydrates, and/or nucleic acids. In some embodiments, the microparticle can include silica microparticles, gold microparticles, graphene microparticles, graphene oxide microparticles, and polystyrene microparticles

In some embodiments, the microparticle can be a non-polymeric particle (e.g. metal particles, quantum dots, ceramics, inorganic materials, bone, etc.).

In some embodiments, the microparticle is selected from n-acetylglucosamine, cyclo-dextrin, polysaccharides copolymers of lactic acid and glycolic acid, starch-borate complexes, polyvinyl alcohol-borate complexes, polypeptides, polyvinyl alcohol, gelled protein solution, soluble potato starch hydrolysate, alginate, cross-linked alginate, starches, dextrans, celluloses, hydroxy-propyl cellulose, chitin, de-acetylated chitin, chitosan, hyaluronic acid and its derivatives, collagen, albumin, gelatin, glycosaminoglycans, polylactic acid (PLA), copolymers of lactic and aminocaproic acid, polyglycolic acid (PGA), poly(lactic-co-glycolic acid) (PLGA), polyoxyethylene-polyoxypropylene block copolymers, poly(DL-lactamide), copolymers of leucine and glutamic acid, polystyrene, polyesters, poly (ortho esters), non-biodegradable polyurethanes, polyureas, poly(vinyl alcohol), polyanhydrides, polyamides, poly (tetrafluoroethylene), poly (ethylene vinyl acetate), polypropylene, polyacrylate, non-biodegradable polycyanoacrylates, polyacryl starch, non-biodegradable polyurethanes, polymethacrylate, poly (methyl methacrylate), polyethylene, polypyrrole, polyanilines, polythiophene, and poly (ethylene oxide).

In some embodiments, the average size of the nanoparticle is between 5 to 500 nm.

In some embodiments, the nanoparticle is selected from the group consisting of albumin-based nanoparticles (e.g., bovine serum albumin, human serum albumin), silica nanoparticles, gold nanoparticles, graphene nanoparticles, graphene oxide nanoparticles, corn zein nanoparticles, polystyrene nanoparticles, fullerenes, endohedral metallofullerenes bucky balls, trimetallic nitride templated endohedral metallofullerenes, single-walled and mutli-walled carbon nanotubes, branched and dendritic carbon nanotubes, gold nanorods, silver nanorods, single-walled and multi-walled boron/nitrate nanotubes, carbon nanotube peapods, carbon nanohorns, carbon nanohorn peapods, liposomes, nanoshells, dendrimers, any nanostructures, microstructures, or their derivatives formed using layer-by-layer processes, self-assembly processes, or polyelectrolytes, microparticles, quantum dots, superparamagnetic nanoparticles, nanorods, cellulose nanoparticles, glass and polymer micro- and nano-spheres, biodegradable PLGA micro- and nano-spheres, gold nanoparticles, silver nanoparticles, carbon nanoparticles, iron nanoparticles, a modified micelle, metal-polyhistidine-DOPE@liposome, metal-polyhistidine-PEG, 4arm-PEG-polyhistidine-metal hydrogels, and sHDL-polyhistidine, and metal-organic framework (MOF) coordination polymer (CP).

In some embodiments, the biodegradable agent is a fiber containing prebiotic agent. In some embodiments, the fiber containing prebiotic agent is selected from epigallocatechin gallate (EGCG), fucoidan, potato starch, oligofructose and inulin.

In some embodiments, the fiber containing prebiotic agent is inulin. Inulin is a polysaccharide belonging to the fructan group. It consists of a beta-2-1-linked chain of fructose molecules, this chain having at its end an alpha-D-glucose unit at the reducing end. Inulin occurs in economically recoverable amounts in various plants such as, for example, chicory roots and dahlia tubers. In addition, inulin has been found for example in Jerusalem artichokes and artichokes. The average chain lengths of the various inulins and their physico-chemical properties differ from plant species to plant species. In some embodiments, the agent is a gel-based inulin formulation. In some embodiments, the agent is a gel-based inulin formulation having an average degree of polymerization at or higher than 20 and at or less than 47. In some embodiments, the fiber containing prebiotic agent is a gel-based inulin formulation having an average degree of polymerization at approximately 28 (e.g., 23, 24, 25, 26, 27, 28, 29, 30, 31, 32, 33). In some embodiments, the gel-based inulin formulation comprises one or more prebiotic compounds selected from a fructo-oligosaccharide, a short-chain fructo-oligosaccharide, an isomalt-oligosaccharide, a transgalacto-oligosaccharide, a pectin, a xylo-oligosaccharide, a chitosan-oligosaccharide, a beta-glucan, an arable gum modified starch, a resistant potato starch, guar gum, bean gum, gelatin, glycerol, a polydextrose, a D-tagatose, an acacia fiber, carob, an oat, and a citrus fiber.

Such compositions are not limited to a particular therapeutic agent.

In some embodiments, the therapeutic agent is an immune checkpoint inhibitor (ICI). In some embodiments, the ICI is capable of binding to, blocking, and/or inhibit the activity of one or more of CTLA-4, PDL1, PDL2, PD1, BTLA, HVEM, TIM3, GAL9, LAG3, VISTA, KIR, 2B4, CD160 and CGEN-15049. In some embodiments, the ICI is selected from Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

In some embodiments, the therapeutic agent is an immunostimulatory agent. In some embodiments, the immunostimulatory agent is selected from anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, inhibitors of IDO, CpG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2. IFN-a, Flt-3L), IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MEDI9197, glucopyranosyl lipid adjuvant (GLA), GLA-SE, CD1d ligands (such as C20:2, OCH, AH04-2, α-galatosylceramide, α-C-galatosylceramide, α-mannosylceramide, α-fructosylceramide, β-galatosylceramide, β-mannosylceramide), STING agonists (e.g. cyclic dinucleotides, including Cyclic [G(3′,5″)pA(3′,5′)p], Cyclic [G(2′,5′)pA(3,5)p], Cyclic [G(2′,5″)pA(2′,5′)p], Cyclic diadenylate monophosphate. Cyclic diguanylate monophosphate), CL401. CL413. CL429, Flagellin. RC529, E6020, imidazoquinoline-based small molecule TLR-7/8a (including its lipidated analogues), virosomes, AS01, AS02, AS03, AS04, AS15, IC31, CAF01, ISCOM. Cytokines (such as GM-CSF, IL-2, IFN-a, Flt-3L), bacterial toxins (such as CT, and LT), cations (such as Zn²⁺, Mn²⁺, Ca²⁺, Fe²⁺, Fe³⁺, Cu²⁺, Ni²⁺, Co²⁺, Pb²⁺, Sn²⁺, Ru²⁺, Au²⁺, Mg²⁺, VO²⁺, Al³⁺, Co³⁺, Cr³⁺, Ga³⁺, Tl³⁺, Ln³⁺, MoO³⁺, Cu⁺, Au⁺, Tl⁺, Ag⁺, Hg²⁺, Pt²⁺, Pb²⁺, Hg²⁺, Cd²⁺, Pd²⁺, Pt⁴⁺, Na⁺, K⁺, and relative phosphate or carbonate salt), any derivative of an immunostimulatory agent, and any combination of immunostimulatory agents.

In some embodiments, the STING agonist is selected from the group consisting of cGAMP, cdiAMP, cdiGMP, cAIMP, 2′3′-cGAMP, 3′3′-cGAMP, c-di-AMP, c-di-GMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP,

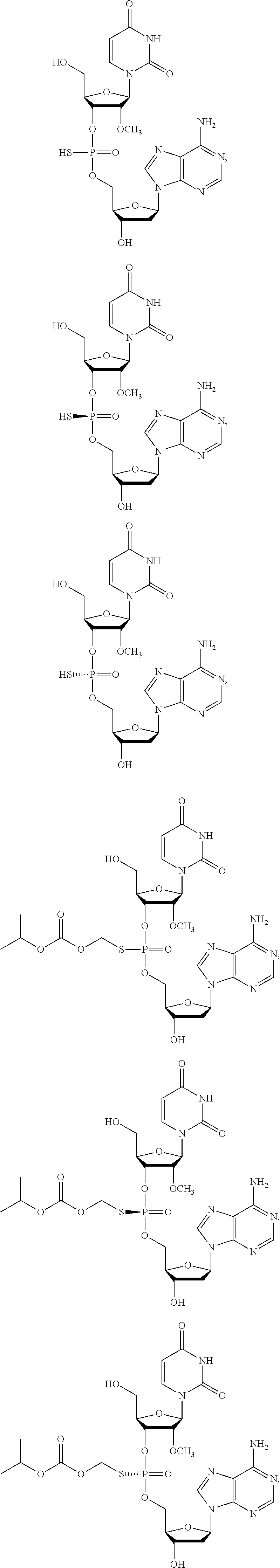

### SB11285 (Spring Bank Pharmaceuticals), Gemcitabine (

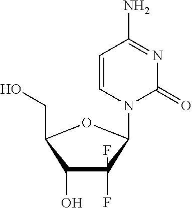

### STING-agonist-C11 (

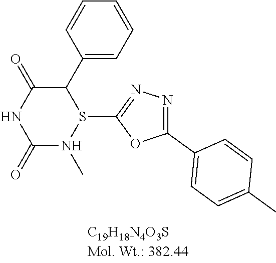

STING agonist-1 (

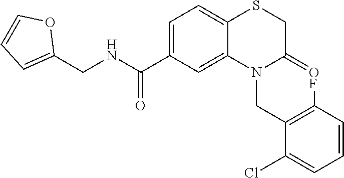

STING agonist G10 (

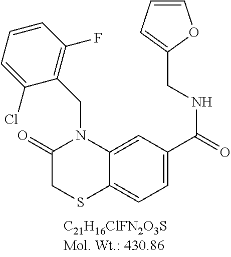 [SMILES: O=C(NCc1ccco1)c1ccc2c(c1)N(Cc1c(F)cccc1Cl)C(=O)CS2]

2′3′-cGAMP, 3′3′-cGAMP, c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2, 2′3′-cGAM(PS)2(Rp/Sp), 2′2′-cGAM(PS)2, 2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2, 2′3′-c-di-AM(PS)2 (Rp,Rp), 2′2′-c-di-AM(PS)2, 3′3′-c-di-AM(PS)2, c-di-AMP Fluorinated, 2′3′-cdAMP Fluorinated, 2′2′-cdAMP Fluorinated, 3′3′-cdAMP Fluorinated, cdGMP, 2′3′-cdGMP, 2′2′-cdGMP, 3′3′-cdGMP, c-di-GM(PS)2, 2′3′-c-di-GM(PS)2, 2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIMP Fluorinated, cAIM(PS)2 Difluor, 3′3′-cAIM(PS)2 Difluor (Rp/Sp), 2′3′-cAIM(PS)2 Difluor, 2′2′-cAIM(PS)2 Difluor, c-di-IMP, 2′3′-cdIMP, 2′2′-cdIMP, 3′3′-cdIMP, c-di-IM(PS)2, 2′3′-c-di-IM(PS)2, 2′2′-c-di-IM(PS)2, 3′3′-c-di-IM(PS)2, c-di-IMP Fluorinated, 2′3′-cdIMP Fluorinated. 2′2″-cdIMP Fluorinated. 3′3′-cdIMP Fluorinated, and amidobenzimidazole (ABZI)-based compounds.

In some embodiments, the therapeutic agent is an immunotherapy. In some embodiments, the immunotherapy is a PD-1 inhibitor such as a PD-1 antibody, a PD-L1 inhibitor such as a PD-L1 antibody, a CTLA-4 inhibitor such as a CTLA-4 antibody, a CSF-1 R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor. In some embodiments, the immunotherapy is a PD-1 inhibitor (e.g., nivolumab, pembrolizumab, pidilizumab, BMS 936559, and MPDL3280A). In some embodiments, the immunotherapy is a PD-L1 inhibitor (e.g., atezolizumab and MED14736). In some embodiments, the immunotherapy is a CTLA-4 inhibitor (e.g., ipilimumab). In some embodiments, the immunotherapy is a CSF-1 R inhibitor (e.g., pexidartinib and AZD6495). In some embodiments, the immunotherapy is an IDO inhibitor (e.g., norharmane, rosmarinic acid, and alpha-methyl-tryptophan). In some embodiments, the immunotherapy is an A1 adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In some embodiments, the immunotherapy is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In some embodiments, the immunotherapy is an A2B adenosine inhibitor (e.g., ATL-801, CVT-6883, MRS-1706, MRS-1754, OSIP-339,391, PSB-603, PSB-0788, or PSB-1115). In some embodiments, the immunotherapy is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In some embodiments, the immunotherapy is an arginase inhibitor (e.g., an arginase antibody, (2s)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapy is an HDAC inhibitor (e.g., valproic acid, SAHA, or romidepsin).

In some embodiments, the therapeutic agent is capable of treating, preventing, and/or ameliorating the symptoms of any type of pancreatitis (e.g., ampicillin, imipenem/cilastatin, ceftriaxone sodium).

In some embodiments, the therapeutic agent is capable of treating, preventing, and/or ameliorating the symptoms of any type of diabetes (e.g., any type of insulin, any type of biguanide (e.g., any type or derivative of metformin), any type of alpha-glucosidase inhibitor (e.g., acarbose, miglitol), any type of dopamine agonist (e.g., bromocriptine), any type of dipeptidyl peptidase-4 inhibitor (e.g., alogliptin (Nesina), alogliptin-metformin (Kazano), alogliptin-pioglitazone (Oseni), linagliptin (Tradjenta), linagliptin-empagliflozin (Glyxambi), linagliptin-metformin (Jentadueto), saxagliptin (Onglyza), saxagliptin-metformin (Kombiglyze XR), sitagliptin (Januvia), sitagliptin-metformin (Janumet and Janumet XR), sitagliptin and simvastatin (Juvisync)), any type of glucagon-like peptide-1 receptor agonist (e.g., albiglutide (Tanzeum), dulaglutide (Trulicity), exenatide (Byetta), exenatide extended-release (Bydureon), liraglutide (Victoza), semaglutide (Ozempic)), any type meglitinide (e.g., nateglinide (Starlix), repaglinide (Prandin), repaglinide-metformin (Prandimet)), any type of sodium-glucose transporter 2 inhibitor (e.g., dapagliflozin (Farxiga), dapagliflozin-metformin (Xigduo XR), canagliflozin (Invokana), canagliflozin-metformin (Invokamet), empagliflozin (Jardiance), empagliflozin-linagliptin (Glyxambi), empagliflozin-metformin (Synjardy), ertugliflozin (Steglatro)), any type of sulfonylurea (e.g., glimepiride (Amaryl), glimepiride-pioglitazone (Duetact), glimepiride-rosiglitazone (Avandaryl), gliclazide, glipizide (Glucotrol), glipizide-metformin (Metaglip), glyburide (DiaBeta, Glynase, Micronase), glyburide-metformin (Glucovance), chlorpropamide (Diabinese), tolazamide (Tolinase), tolbutamide (Orinase, Tol-Tab)), any type of thiazolidinedione (e.g., rosiglitazone (Avandia), rosiglitazone-glimepiride (Avandaryl), rosiglitazone-metformin (Amaryl M), pioglitazone (Actos), pioglitazone-alogliptin (Oseni), pioglitazone-glimepiride (Duetact), and pioglitazone-metformin (Actoplus Met, Actoplus Met XR)).

In some embodiments, the therapeutic agent is capable of treating, preventing, and/or ameliorating the symptoms of any type of pancreatic cancer (e.g., Capecitabine (Xeloda), Erlotinib (Tarceva), Fluorouracil (5-FU), Gemcitabine (Gemzar), Irinotecan (Camptosar), Leucovorin (Wellcovorin), Nab-paclitaxel (Abraxane), Nanoliposomal irinotecan (Onivyde), Oxaliplatin (Eloxatin)).

In some embodiments, the therapeutic agent is capable of treating, preventing, and/or ameliorating the symptoms of any type of cholangitis (e.g., ursodeoxycholic acid, obeticholic acid, fibrates, budesonide).

In some embodiments, the therapeutic agent is capable of treating, preventing, and/or ameliorating the symptoms of any type of bile duct cancer (e.g., 5-fluorouracil (5-FU), Gemcitabine (Gemzar), Cisplatin (Platinol), Capecitabine (Xeloda), Oxaliplatin (Eloxatin)).

In some embodiments, the therapeutic agent is capable of treating, preventing, and/or ameliorating the symptoms of any type of gallstone (e.g., ursodiol link (Actigall) and chenodiol link (Chenix)).

In some embodiments, the therapeutic agent is capable of treating, preventing, and/or ameliorating the symptoms of any type of gall bladder cancer (e.g., 5-fluorouracil (5-FU), Gemcitabine (Gemzar), Cisplatin (Platinol), Capecitabine (Xeloda), Oxaliplatin (Eloxatin)).

In some embodiments, the therapeutic agent is capable of treating, preventing, and/or ameliorating the symptoms of any type of hepatitis (e.g., any type of antiviral medication (e.g., entecavir (Baraclude), tenofovir (Viread), lamivudine (Epivir), adefovir (Hepsera), telbivudine (Tyzeka)), interferon alfa-2b).

In some embodiments, the therapeutic agent is capable of treating, preventing, and/or ameliorating the symptoms of any type of cholangitis (e.g., ursodeoxycholic acid (UDCA), obeticholic acid, fibrates, budesonide).

In some embodiments, the therapeutic agent is capable of treating, preventing, and/or ameliorating the symptoms of any type of liver cancer (e.g., Atezolizumab, Avastin (Bevacizumab), Bevacizumab, Cabometyx (Cabozantinib-S-Malate), Cabozantinib-S-Malate, Cyramza (Ramucirumab), Keytruda (Pembrolizumab), Lenvatinib Mesylate, Lenvima (Lenvatinib Mesylate), Nexavar (Sorafenib Tosylate), Nivolumab, Opdivo (Nivolumab), Pemazyre (Pemigatinib), Pembrolizumab, Pemigatinib, Ramucirumab, Regorafenib, Sorafenib Tosylate, Stivarga (Regorafenib), Tecentriq (Atezolizumab)).

In some embodiments, the therapeutic agent is any type or kind of chemotherapeutic agent. In some embodiments, the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (TAXOL), pilocarpine, prochloroperazine, rituximab, tamoxifen, taxol, topotecan hydrochloride, trastuzumab, vinblastine, vincristine and vinorelbine tartrate.

In some embodiments, the composition or biodegradable agent further comprises a diagnostic agent. Such embodiments are not limited to a particular type of diagnostic agent.

In some embodiments, the diagnostic agent is an antibody. In some embodiments, the antibody is 3F8, Abagovomab, Abciximab, Abituzumab, Abrilumab, Acritumomab, Actoxumab, Adalimumab, Adalimumab-atto, Adecatumumab, Ado-trastuzumab emtansine, Aducanumab, Afasevikumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, Crenezumab, Crotedumab, CR6261, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Inebilizumab, Infliximab, Infliximab-dyyb, Intetumumab, Inolimomab, Inotuzumab ozogamicin, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab emtansine, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Mapatumumab, Margetuximab, Maslimomab, Mavrilimumab, Matuzumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tildrakizumab, Tigatuzumab, Timolumab, Tisotumab vedotin, TNX-650), Tocilizumab, Toralizumab, Tosatoxumab, Tositumomab, Tovetumab, Tralokinumab, Trastuzumab, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, or Zolimomab aritox.

In some embodiments, the diagnostic agent is Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase Clostridium histolyticum, Darbepoetin alfa, Denileukin diftitox, Dornase alfa, Dulaglutide, Ecallantide, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1 a, Interferon beta-1 b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a. Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, or Ziv-aflibercept.

In some embodiments, the diagnostic agent is tuberculin purified protein derivative, hyrotropin alpha, secretin, soluble transferrin receptor, troponin, B-type natriuretic peptide, iobenguane I 123, florbetapir F 18, perflutren, gadoterate meglumine, florbetaben F 18, flutemetamol F 18, gadoterate meglumine, isosulfan blue, regadenoson, technetium Tc 99m tilmanocept, florbetaben F 18, perflutren, regadenoson, or flutemetamol F 18.

In some embodiments, the composition or biodegradable agent further comprises an agent capable of relaxing (e.g., opening, dilating) the hepatopancreatic anatomical region (e.g., hepatopancreatic ampulla, hepatopancreatic duct, Ampulla of Vater, Sphincter of Oddi) in a subject. Such embodiments are not limited to a particular type of agent capable of relaxing (e.g., opening, dilating) the hepatopancreatic anatomical region (e.g., hepatopancreatic ampulla, hepatopancreatic duct, Ampulla of Vater, Sphincter of Oddi) in a subject. In some embodiments, the agent capable of relaxing (e.g., opening, dilating) the hepatopancreatic anatomical region is selected from nitroglycerin, scopolamine butylbromide, hymecromone (7-hydroxy-4-methylumbelliferone), botulinum toxin, vasoactive intestinal polypeptide, cholecystokinin-8, and secretin.

In some embodiments, the composition or biodegradable agent further comprises a diagnostic agent and an agent capable of relaxing (e.g., opening, dilating) the hepatopancreatic anatomical region (e.g., hepatopancreatic ampulla, hepatopancreatic duct, Ampulla of Vater, Sphincter of Oddi) in a subject.

In certain embodiments, the present invention provides methods for treating, preventing, ameliorating the symptoms of, and/or diagnosing a condition in a subject, the method comprising administering to a subject a pharmaceutically effective amount of a composition comprising biodegradable agent associated with (e.g., complexed, conjugated, encapsulated, absorbed, adsorbed, admixed) one or more of a therapeutic agent, a diagnostic agent, and an agent capable of relaxing (e.g., opening, dilating) the hepatopancreatic anatomical region (e.g., hepatopancreatic ampulla, hepatopancreatic duct, Ampulla of Vater, Sphincter of Oddi).

In some embodiments, the composition is co-administered with an agent capable of relaxing (e.g., opening, dilating) the hepatopancreatic anatomical region (e.g., hepatopancreatic ampulla, hepatopancreatic duct, Ampulla of Vater, Sphincter of Oddi) in the subject. Such methods are not limited to specific agents capable of relaxing (e.g., opening, dilating) the hepatopancreatic anatomical region (e.g., hepatopancreatic ampulla, hepatopancreatic duct, Ampulla of Vater, Sphincter of Oddi). In some embodiments, the agent capable of relaxing (e.g., opening) the hepatopancreatic anatomical region (e.g., hepatopancreatic ampulla, hepatopancreatic duct, Ampulla of Vater, Sphincter of Oddi) is selected from nitroglycerin, scopolamine butylbromide, hymecromone (7-hydroxy-4-methylumbelliferone), botulinum toxin, vasoactive intestinal polypeptide, cholecystokinin-8, and secretin.

Such methods are not limited to a particular type of subject. In some embodiments, the subject is a mammalian subject. In some embodiments, the subject is a human subject.

Such methods are not limited to a particular manner of administration of the composition comprising biodegradable agent associated with (e.g., complexed, conjugated, encapsulated, absorbed, adsorbed, admixed) one or more of a therapeutic agent, a diagnostic agent, and an agent capable of relaxing (e.g., opening, dilating) the hepatopancreatic anatomical region (e.g., hepatopancreatic ampulla, hepatopancreatic duct, Ampulla of Vater, Sphincter of Oddi). In some embodiments, the composition is administered orally. In some embodiments, the composition is administered by oral gavage. In some embodiments, the composition is administered via transpapillary administration. In some embodiments, the composition is administered intratumorally. In some embodiments, the composition is administered intravenously. In some embodiments, the composition is administered subcutaneously. In some embodiments, the composition is administered intramuscularly.

Such methods are not limited to treating, preventing, and/or ameliorating the symptoms of a particular condition.

In some embodiments, the condition is any disease related to the pancreas (e.g., any type of pancreatic cancer, pancreatitis, diabetes). In some embodiments, the condition is any disease related to the bile duct (e.g., cholangitis, bile duct leaks, biliary stricture, biliary stones, bile duct cancer (e.g., cholangiocarcinoma). In some embodiments, the condition is any disease related to the gall bladder (e.g., gall stones, gall bladder carcinoma). In some embodiments, the condition is any disease related to the liver (e.g., hepatitis, cholangitis, biliary atresia, alpha-1 antitrypsin deficiency, alagille syndrome, progressive familial intrahepatic cholestasis, Langerhans cell histiocytosis, hepatic hemangioma, polycystic liver disease, liver cancer).

The present invention also provides kits comprising a comprising biodegradable agents (e.g., microparticles, nanoparticles) associated with (e.g., complexed, conjugated, encapsulated, absorbed, adsorbed, admixed) therapeutic agents, and one or more of an agent capable of relaxing the Sphincter of Oddi, and additional therapeutic agents capable of treating, preventing and/or ameliorating the symptoms of a disease or condition related to the pancreatic/biliary anatomical region.

### BRIEF DESCRIPTION OF THE DRAWINGS

FIG. 1A-B: (a) ⁶⁸Ga-labeled microparticles: (b) orthotopic murine pancreatic tumor mouse model.

FIG. 2: TEM images of SiO₂ NPs and polystyrene MPs.

FIG. 3: Biodistribution of ⁶⁸Ga labeled NPs or MPs in naïve and pancreatic tumor-bearing mice.

FIG. 4: Biodistribution of ⁶⁸Ga labeled MPs with or without butylscopolamine.

FIG. 5: Biodistribution of ⁶⁸Ga labeled MPs with or without nitroglycerin, or butyl scopolamine, or CCK.

FIG. 6A-C: (a) SEM image of PSMP and accumulation of PSMP in (b) pancreas and (c) gallbladder with or without administration of scopolamine or nitroglycerin.

FIG. 7A-C: (a) SEM image of Nano-calcium MP and accumulation of Nano-calcium MP in (b) pancreas and (c) gallbladder with or without administration of scopolamine.

### DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of and “consisting of those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The transitional term “comprising,” which is synonymous with “including,” “containing,” or “characterized by,” is inclusive or open-ended and does not exclude additional, unrecited elements or method steps. By contrast, the transitional phrase “consisting of excludes any element, step, or ingredient not specified in the claim. The transitional phrase “consisting essentially of limits the scope of a claim to the specified materials or steps “and those that do not materially affect the basic and novel characteristic(s)” of the claimed invention.

A “subject” can be a vertebrate, a mammal, or a human. Mammals include, but are not limited to, farm animals, sport animals, pets, primates, mice and rats. In one aspect, a subject is a human.

As used herein, the term “complexed” as used herein relates to the non-covalent interaction of a metabolite (as described herein) with a biodegradable agent (e.g., nanoparticle and/or microparticle).

As used herein, the term “conjugated” as used herein indicates a covalent bond association between a metabolite (as described herein) with a biodegradable agent (e.g., nanoparticle and/or microparticle).

As used herein, the term “encapsulated” refers to the location of a metabolite (as described herein) that is enclosed or completely contained within the inside of a biodegradable agent (e.g., nanoparticle and/or microparticle).

As used herein, the term “absorbed” refers to metabolite (as described herein) that is taken into and stably retained in the interior, that is, internal to the outer surface, of a biodegradable agent (e.g., nanoparticle and/or microparticle).

As used herein, the term “adsorbed” refers to the attachment of a metabolite (as described herein) to the external surface of a biodegradable agent (e.g., nanoparticle and/or microparticle). Such adsorption preferably occurs by electrostatic attraction. Electrostatic attraction is the attraction or bonding generated between two or more oppositely charged or ionic chemical groups. Generally, the adsorption is typically reversible.

As used herein, the term “admixed” refers to a metabolite (as described herein) that is dissolved, dispersed, or suspended in a biodegradable agent (e.g., nanoparticle and/or microparticle). In some cases, the metabolite may be uniformly admixed in the biodegradable agent (e.g., nanoparticle and/or microparticle).

Unless otherwise defined, all technical terms used herein have the same meaning as commonly understood by one of ordinary skill in the art to which this disclosure belongs.

### DETAILED DESCRIPTION

In experiments conducted during the course of developing embodiments for the present invention determined that micro-sized or nano-sized particles (MPs) given via oral route can directly enter the pancreas. When such microparticles or nanoparticles are co-administered with commercial drugs known to relax the hepatopancreatic anatomical region (e.g., hepatopancreatic ampulla, hepatopancreatic duct, Ampulla of Vater, Sphincter of Oddi), this led to even further improvement in the accumulation of microparticles or nanoparticles in the pancreas.

Based on murine studies described herein, it was envisioned that chemotherapeutic-loaded micro or nano particles of a certain size range (e.g., 0.1 to 100 μm in diameter) can effectively deliver chemotherapeutic agents to the pancreas after oral administration, while significantly decreasing the systemic exposure of chemo-drugs and reducing off-target toxicity. Once the chemo-drugs loaded microparticles or nanoparticles enter the pancreas, they diffuse throughout the whole pancreas and remain in pancreas for hours to days. In the meantime, the payload can be slowly released within the pancreas, thus greatly improving the bioavailability and pharmacokinetics of chemo-drugs.

Accordingly, the present invention relates to biodegradable agents associated with (e.g., complexed, conjugated, encapsulated, absorbed, adsorbed, admixed) one or more of a therapeutic agent (e.g., configured for treating, preventing or ameliorating various types of disorders), a diagnostic agent, and an agent capable of relaxing (e.g., opening, dilating) the hepatopancreatic anatomical region, as well as systems and methods utilizing such compositions (e.g., in diagnostic and/or therapeutic settings (e.g., treating pancreatic cancers)).

In certain embodiments, the present invention provides compositions comprising a biodegradable agent (e.g., a microparticle or a nanoparticle) (e.g., particle having a diameter between approximately 0.1 to 100 μm) associated with one or more of a therapeutic agent, a diagnostic agent, and an agent capable of relaxing (e.g., opening, dilating) the hepatopancreatic anatomical region. For example, in some embodiments, the present invention provides compositions comprising biodegradable agents (e.g., microparticles or a nanoparticles) associated with (e.g., complexed, conjugated, encapsulated, absorbed, adsorbed, admixed) one or more of a a diagnostic agent, an agent capable of relaxing (e.g., opening, dilating) the hepatopancreatic anatomical region, and a therapeutic agent (e.g., agents capable of treating and/or ameliorating the symptoms of conditions associated with the pancreas (e.g., pancreatic cancer, pancreatitis, diabetes), conditions associated with the bile duct (e.g., cancer associated with the bile duct, cholangitis), conditions associated with the liver (e.g., liver cancer, hepatitis), conditions associated with the gall bladder (e.g., gall bladder cancer, gall stones, cholecystitis).

In some embodiments, the size of the diameter of the biodegradable agent is between approximately 0.1 to 100 μm (e.g., 0.001 μm, 0.01 μm, 0.1 μm, 0.5 μm, 1 μm, 1.5 μm, 1.999 μm, 2 μm, 5 μm, 10 μm, 15 μm, 19 μm, 19.9999 μm, 20 μm, 20.01 μm, 20.5 μm, 21 μm, 25 μm, 30 μm, 50 μm, 70 μm, 80 μm, 100 μm, 101 μm, 105 μm, 115 μm) (e.g., 0.001 to 115 μm, 0.1 to 105 μm, 0.5 to 101 μm, 1 to 100 μm, 1.9999 to 80 μm, 2 to 70 μm, 5 to 50 μm, 10 to 30 μm, 15 to 25 μm, 19 to 21 μm, 19.9999 to 20.5 μm, 20 to 20.01 μm). In some embodiments, the size of the diameter of the biodegradable agent is between approximately 5 to 20 μm.

In some embodiments, biodegradable agent is a microparticle or nanoparticle.

In some embodiments, the size of the microparticle is between 0.1 microns to 100 microns.

In some embodiments, the microparticle is a polystyrene microparticle.

In some embodiments, the microparticle is a bovine serum albumin microparticle. In some embodiments, the microparticle is a human serum albumin microparticle. In some embodiments, the microparticle is a bovine serum albumin microparticle.

In some embodiments, the microparticle is a corn zein microparticle.

In some embodiments, the microparticle is a biocompatible polymer selected from the group consisting of poly (lactide), poly(lactide-co-glycolide)s, poly (caprolactone), polycarbonates, polyamides, polyanhydrides, polyamino acids, polyortho esters, polyacetals, polycyanoacrylates, degradable polyurethanes, polyacrylates, polymers of ethylene-vinyl acetate and other acyl substituted cellulose acetates and derivatives thereof, polysaccharide, non-erodible polyurethanes, polystyrenes, polyvinyl chloride, polyvinyl fluoride, poly (vinyl imidazole), chlorosulphonated polyolifins, polyethylene oxide, and copolymers and mixtures thereof.

In some embodiments, the microparticle can comprise polyethylenes, polycarbonates, polyanhydrides, polyhydroxyacids, polypropylfumerates, poly caprolactones, polyamides, polyacetals, polyethers, polyesters, poly(orthoesters), polycyanoacrylates, polyvinyl alcohols, polyurethanes, polyphosphazenes, polyacrylates, polymethacrylates, polyureas, polystyrenes, and/or polyamines. In some embodiments, a polymeric matrix may comprise poly(lactic-co-glycolic acid) (PLGA), polyethylene glycol (PEG), and/or copolymers thereof. In some embodiments, a polymeric particle can comprise dendrimers, proteins, carbohydrates, and/or nucleic acids. In some embodiments, the microparticle can include silica microparticles, gold microparticles, graphene microparticles, graphene oxide microparticles, and polystyrene microparticles

In some embodiments, the microparticle can be a non-polymeric particle (e.g. metal particles, quantum dots, ceramics, inorganic materials, bone, etc.).

In some embodiments, the microparticle is selected from n-acetylglucosamine, cyclo-dextrin, polysaccharides copolymers of lactic acid and glycolic acid, starch-borate complexes, polyvinyl alcohol-borate complexes, polypeptides, polyvinyl alcohol, gelled protein solution, soluble potato starch hydrolysate, alginate, cross-linked alginate, starches, dextrans, celluloses, hydroxy-propyl cellulose, chitin, de-acetylated chitin, chitosan, hyaluronic acid and its derivatives, collagen, albumin, gelatin, glycosaminoglycans, polylactic acid (PLA), copolymers of lactic and aminocaproic acid, polyglycolic acid (PGA), poly(lactic-co-glycolic acid) (PLGA), polyoxyethylene-polyoxypropylene block copolymers, poly(DL-lactamide), copolymers of leucine and glutamic acid, polystyrene, polyesters, poly (ortho esters), non-biodegradable polyurethanes, polyureas, poly (vinyl alcohol), polyanhydrides, polyamides, poly (tetrafluoroethylene), poly (ethylene vinyl acetate), polypropylene, polyacrylate, non-biodegradable polycyanoacrylates, polyacryl starch, non-biodegradable polyurethanes, polymethacrylate, poly (methyl methacrylate), polyethylene, polypyrrole, polyanilines, polythiophene, and poly (ethylene oxide).

In some embodiments, the average size of the nanoparticle is between 5 to 500 nm.

In some embodiments, the nanoparticle is selected from the group consisting of albumin-based nanoparticles (e.g., bovine serum albumin, human serum albumin), silica nanoparticles, gold nanoparticles, graphene nanoparticles, graphene oxide nanoparticles, corn zein nanoparticles, polystyrene nanoparticles, fullerenes, endohedral metallofullerenes bucky balls, trimetallic nitride templated endohedral metallofullerenes, single-walled and mutli-walled carbon nanotubes, branched and dendritic carbon nanotubes, gold nanorods, silver nanorods, single-walled and multi-walled boron/nitrate nanotubes, carbon nanotube peapods, carbon nanohorns, carbon nanohorn peapods, liposomes, nanoshells, dendrimers, any nanostructures, microstructures, or their derivatives formed using layer-by-layer processes, self-assembly processes, or polyelectrolytes, microparticles, quantum dots, superparamagnetic nanoparticles, nanorods, cellulose nanoparticles, glass and polymer micro- and nano-spheres, biodegradable PLGA micro- and nano-spheres, gold nanoparticles, silver nanoparticles, carbon nanoparticles, iron nanoparticles, a modified micelle, metal-polyhistidine-DOPE@liposome, metal-polyhistidine-PEG, 4arm-PEG-polyhistidine-metal hydrogels, and sHDL-polyhistidine, and metal-organic framework (MOF) coordination polymer (CP).

In some embodiments, the biodegradable agent is a fiber containing prebiotic agent. In some embodiments, the fiber containing prebiotic agent is selected from epigallocatechin gallate (EGCG), fucoidan, potato starch, oligofructose and inulin.

In some embodiments, the fiber containing prebiotic agent is inulin. Inulin is a polysaccharide belonging to the fructan group. It consists of a beta-2-1-linked chain of fructose molecules, this chain having at its end an alpha-D-glucose unit at the reducing end. Inulin occurs in economically recoverable amounts in various plants such as, for example, chicory roots and dahlia tubers. In addition, inulin has been found for example in Jerusalem artichokes and artichokes. The average chain lengths of the various inulins and their physico-chemical properties differ from plant species to plant species. In some embodiments, the agent is a gel-based inulin formulation. In some embodiments, the agent is a gel-based inulin formulation having an average degree of polymerization at or higher than 20 and at or less than 47. In some embodiments, the fiber containing prebiotic agent is a gel-based inulin formulation having an average degree of polymerization at approximately 28 (e.g., 23, 24, 25, 26, 27, 28, 29, 30, 31, 32, 33). In some embodiments, the gel-based inulin formulation comprises one or more prebiotic compounds selected from a fructo-oligosaccharide, a short-chain fructo-oligosaccharide, an isomalt-oligosaccharide, a transgalacto-oligosaccharide, a pectin, a xylo-oligosaccharide, a chitosan-oligosaccharide, a beta-glucan, an arable gum modified starch, a resistant potato starch, guar gum, bean gum, gelatin, glycerol, a polydextrose, a D-tagatose, an acacia fiber, carob, an oat, and a citrus fiber.

Such compositions are not limited to a particular therapeutic agent.

In some embodiments, the therapeutic agent is an immune checkpoint inhibitor (ICI). In some embodiments, the ICI is capable of binding to, blocking, and/or inhibit the activity of one or more of CTLA-4, PDL1, PDL2, PD1, BTLA, HVEM, TIM3, GAL9, LAG3, VISTA, KIR, 2B4, CD160 and CGEN-15049. In some embodiments, the ICI is selected from Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

In some embodiments, the therapeutic agent is an immunostimulatory agent. In some embodiments, the immunostimulatory agent is selected from anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO. In some embodiments, the therapeutic agent is an immunostimulatory agent. In some embodiments, the immunostimulatory agent is selected from anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, inhibitors of IDO, CpG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2, IFN-α. Flt-3L), IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MEDI9197, glucopyranosyl lipid adjuvant (GLA), GLA-SE, CD1d ligands (such as C20:2, OCH, AH04-2, α-galatosylceramide, α-C-galatosylceramide, α-mannosylceramide, α-fructosylceramide. β-galatosylceramide. β-mannosylceramide), STING agonists (e.g. cyclic dinucleotides, including Cyclic [G(3′,5′)pA(3′,5′)p], Cyclic [G(2′,5″)pA(3′,5′)p], Cyclic [G(2′,5′)pA(2′,5′)p], Cyclic diadenylate monophosphate, Cyclic diguanylate monophosphate). CL401, CL413, CL429, Flagellin, RC529. E6020, imidazoquinoline-based small molecule TLR-7/8a (including its lipidated analogues), virosomes, AS01, AS02. AS03, AS04, AS15. IC31, CAF01, ISCOM, Cytokines (such as GM-CSF, IL-2, IFN-a, Flt-3L), bacterial toxins (such as CT, and LT), cations (such as Zn²⁺, Mn²⁺, Ca²⁺, Fe²⁺, Fe³⁺, Cu²⁺, Ni²⁺, Co²⁺, Pb²⁺, Sn²⁺, Ru²⁺, Au²⁺, Mg²⁺, VO²⁺, Al³⁺, Co³⁺, Cr³⁺, Ga³⁺, Tl³⁺, Ln³⁺, MoO³⁺, Cu⁺, Au⁺, Tl⁺, Ag⁺, Hg²⁺, Pt²⁺, Pb²⁺, Hg²⁺, Cd²⁺, Pd²⁺, Pt⁴⁺, Na⁺, K⁺, and relative phosphate or carbonate salt), any derivative of an immunostimulatory agent, and any combination of immunostimulatory agents.

In some embodiments, the STING agonist is selected from the group consisting of cGAMP, cdiAMP, cdiGMP, cAIMP, 2′3′-cGAMP, 3′3′-cGAMP, c-di-AMP, c-di-GMP, cAIMP Difluor, cAIM(PS)2, Difluor (Rp/Sp), 2′2-cGAMP, 2′3″-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated. 2′3′-c-di-GMP, c-di-IMP,

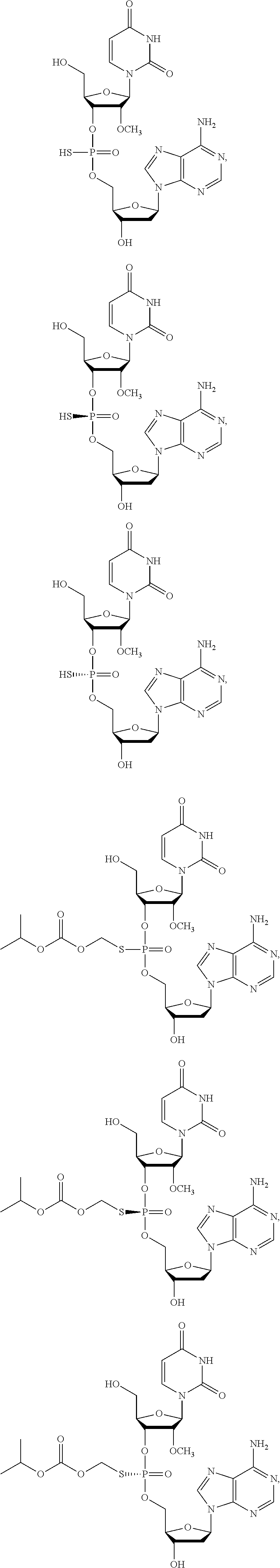

### SB11285 (Spring Bank Pharmaceuticals). Gemcitabine (

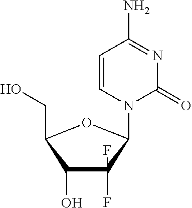

### STING-agonist-C11

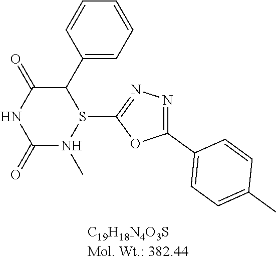

STING agonist-1 (

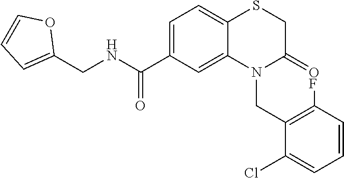

STING agonist G10 (

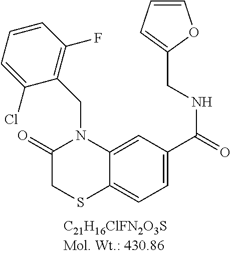 [SMILES: O=C(NCc1ccco1)c1ccc2c(c1)N(Cc1c(F)cccc1Cl)C(=O)CS2]

2′3′-cGAMP, 3′3′-cGAMP, c-di-AMP, c-di-GMP, CAIMP, cAIMP Difluor, cAIM(PS)2, Difluor (Rp/Sp), 2′2′-cGAMP, 2′3′-cGAM(PS)2 (Rp/Sp). 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2, 2′3′-cGAM(PS)2(Rp/Sp), 2′2′-cGAM(PS)2, 2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2, 2′3′-c-di-AM(PS)2 (Rp,Rp), 2′2′-c-di-AM(PS)2, 3′3′-c-di-AM(PS)2, c-di-AMP Fluorinated, 2′3′-cdAMP Fluorinated, 2′2′-cdAMP Fluorinated. 3′3′-cdAMP Fluorinated, cdGMP. 2′3′-cdGMP, 2′2′-cdGMP. 3′3′-cdGMP, c-di-GM(PS)2, 2′3′-c-di-GM(PS)2, 2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIMP Fluorinated, cAIM(PS)2 Difluor, 3′3′-cAIM(PS)2 Difluor (Rp/Sp), 2′3′-cAIM(PS)2 Difluor, 2′2′-cAIM(PS)2 Difluor, c-di-IMP, 2′3″-cdIMP, 2′2′-cdIMP, 3′3′-cdIMP, c-di-IM(PS)2, 2′3′-c-di-IM(PS)2, 2′2′-c-di-IM(PS)2, 3′3′-c-di-IM(PS)2, c-di-IMP Fluorinated, 2′3′-cdIMP Fluorinated, 2′2″-cdIMP Fluorinated, 3′3′-cdIMP Fluorinated, and amidobenzimidazole (ABZI)-based compounds.

In some embodiments, the therapeutic agent is an immunotherapy. In some embodiments, the immunotherapy is a PD-1 inhibitor such as a PD-1 antibody, a PD-L1 inhibitor such as a PD-L1 antibody, a CTLA-4 inhibitor such as a CTLA-4 antibody, a CSF-1 R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor. In some embodiments, the immunotherapy is a PD-1 inhibitor (e.g., nivolumab, pembrolizumab, pidilizumab, BMS 936559, and MPDL3280A). In some embodiments, the immunotherapy is a PD-L1 inhibitor (e.g., atezolizumab and MED14736). In some embodiments, the immunotherapy is a CTLA-4 inhibitor (e.g., ipilimumab). In some embodiments, the immunotherapy is a CSF-1 R inhibitor (e.g., pexidartinib and AZD6495). In some embodiments, the immunotherapy is an IDO inhibitor (e.g., norharmane, rosmarinic acid, and alpha-methyl-tryptophan). In some embodiments, the immunotherapy is an A1 adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In some embodiments, the immunotherapy is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In some embodiments, the immunotherapy is an A2B adenosine inhibitor (e.g., ATL-801, CVT-6883, MRS-1706, MRS-1754, OSIP-339,391, PSB-603, PSB-0788, or PSB-1115). In some embodiments, the immunotherapy is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In some embodiments, the immunotherapy is an arginase inhibitor (e.g., an arginase antibody, (2s)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapy is an HDAC inhibitor (e.g., valproic acid, SAHA, or romidepsin).

In some embodiments, the therapeutic agent is capable of treating, preventing, and/or ameliorating the symptoms of any type of pancreatitis (e.g., ampicillin, imipenem/cilastatin, ceftriaxone sodium).

In some embodiments, the therapeutic agent is capable of treating, preventing, and/or ameliorating the symptoms of any type of diabetes (e.g., any type of insulin, any type of biguanide (e.g., any type or derivative of metformin), any type of alpha-glucosidase inhibitor (e.g., acarbose, miglitol), any type of dopamine agonist (e.g., bromocriptine), any type of dipeptidyl peptidase-4 inhibitor (e.g., alogliptin (Nesina), alogliptin-metformin (Kazano), alogliptin-pioglitazone (Oseni), linagliptin (Tradjenta), linagliptin-empagliflozin (Glyxambi), linagliptin-metformin (Jentadueto), saxagliptin (Onglyza), saxagliptin-metformin (Kombiglyze XR), sitagliptin (Januvia), sitagliptin-metformin (Janumet and Janumet XR), sitagliptin and simvastatin (Juvisync)), any type of glucagon-like peptide-1 receptor agonist (e.g., albiglutide (Tanzeum), dulaglutide (Trulicity), exenatide (Byetta), exenatide extended-release (Bydureon), liraglutide (Victoza), semaglutide (Ozempic)), any type meglitinide (e.g., nateglinide (Starlix), repaglinide (Prandin), repaglinide-metformin (Prandimet)), any type of sodium-glucose transporter 2 inhibitor (e.g., dapagliflozin (Farxiga), dapagliflozin-metformin (Xigduo XR), canagliflozin (Invokana), canagliflozin-metformin (Invokamet), empagliflozin (Jardiance), empagliflozin-linagliptin (Glyxambi), empagliflozin-metformin (Synjardy), ertugliflozin (Steglatro)), any type of sulfonylurea (e.g., glimepiride (Amaryl), glimepiride-pioglitazone (Duetact), glimepiride-rosiglitazone (Avandaryl), gliclazide, glipizide (Glucotrol), glipizide-metformin (Metaglip), glyburide (DiaBeta, Glynase, Micronase), glyburide-metformin (Glucovance), chlorpropamide (Diabinese), tolazamide (Tolinase), tolbutamide (Orinase, Tol-Tab)), any type of thiazolidinedione (e.g., rosiglitazone (Avandia), rosiglitazone-glimepiride (Avandaryl), rosiglitazone-metformin (Amaryl M), pioglitazone (Actos), pioglitazone-alogliptin (Oseni), pioglitazone-glimepiride (Duetact), and pioglitazone-metformin (Actoplus Met, Actoplus Met XR)).

In some embodiments, the therapeutic agent is capable of treating, preventing, and/or ameliorating the symptoms of any type of pancreatic cancer (e.g., Capecitabine (Xeloda), Erlotinib (Tarceva), Fluorouracil (5-FU), Gemcitabine (Gemzar), Irinotecan (Camptosar), Leucovorin (Wellcovorin), Nab-paclitaxel (Abraxane), Nanoliposomal irinotecan (Onivyde), Oxaliplatin (Eloxatin)).

In some embodiments, the therapeutic agent is capable of treating, preventing, and/or ameliorating the symptoms of any type of cholangitis (e.g., ursodeoxycholic acid, obeticholic acid, fibrates, budesonide).

In some embodiments, the therapeutic agent is capable of treating, preventing, and/or ameliorating the symptoms of any type of bile duct cancer (e.g., 5-fluorouracil (5-FU), Gemcitabine (Gemzar), Cisplatin (Platinol), Capecitabine (Xeloda), Oxaliplatin (Eloxatin)).

In some embodiments, the therapeutic agent is capable of treating, preventing, and/or ameliorating the symptoms of any type of gallstone (e.g., ursodiol link (Actigall) and chenodiol link (Chenix)).

In some embodiments, the therapeutic agent is capable of treating, preventing, and/or ameliorating the symptoms of any type of gall bladder cancer (e.g., 5-fluorouracil (5-FU), Gemcitabine (Gemzar), Cisplatin (Platinol), Capecitabine (Xeloda), Oxaliplatin (Eloxatin)).

In some embodiments, the therapeutic agent is capable of treating, preventing, and/or ameliorating the symptoms of any type of hepatitis (e.g., any type of antiviral medication (e.g., entecavir (Baraclude), tenofovir (Viread), lamivudine (Epivir), adefovir (Hepsera), telbivudine (Tyzeka)), interferon alfa-2b).

In some embodiments, the therapeutic agent is capable of treating, preventing, and/or ameliorating the symptoms of any type of cholangitis (e.g., ursodeoxycholic acid (UDCA), obeticholic acid, fibrates, budesonide).

In some embodiments, the therapeutic agent is capable of treating, preventing, and/or ameliorating the symptoms of any type of liver cancer (e.g., Atezolizumab, Avastin (Bevacizumab), Bevacizumab, Cabometyx (Cabozantinib-S-Malate), Cabozantinib-S-Malate, Cyramza (Ramucirumab), Keytruda (Pembrolizumab), Lenvatinib Mesylate, Lenvima (Lenvatinib Mesylate), Nexavar (Sorafenib Tosylate), Nivolumab, Opdivo (Nivolumab), Pemazyre (Pemigatinib), Pembrolizumab, Pemigatinib, Ramucirumab, Regorafenib, Sorafenib Tosylate, Stivarga (Regorafenib), Tecentriq (Atezolizumab)).

In some embodiments, the therapeutic agent is any type or kind of chemotherapeutic agent. Examples include, but are not limited to, alkylating agents (e.g., chlorambucil, cyclophosphamide, ccnu, melphalan, procarbazine, thiotepa, bcnu, and busulfan), antimetabolites (e.g., 6-mercaptopurine and 5-fluorouracil), anthracyclines (e.g., daunorubicin, doxorubicin, idarubicin, epirubicin, and mitoxantrone), antitumor antibiotics (e.g., bleomycin), monoclonal antibodies (e.g., alemtuzumab, bevacizumab, cetuximab, gemtuzumab, ibritumomab, panitumumab, rituximab, tositumomab, and trastuzumab), platinums (e.g., cisplatin, oxaliplatin, and carboplatin), plant alkaloids (e.g., vincristine), topoisomerase I or II inhibitors (e.g., irinotecan, topotecan, amsacrine, etoposide, etoposide phosphate, and teniposide), vinca alkaloids (e.g., vincristine, vinblastine, vinorelbine, and vindesine), taxanes (e.g., paclitaxel and docetaxel), epipodophyllotoxins (e.g., etoposide and teniposide), nucleoside analogs, and angiogenesis inhibitors (e.g., Avastin (beracizumab), a humanized monoclonal antibody specific for VEGF-A).

Examples of glutathione antagonists include but are not limited to buthionine sulfoximine, cyclophosphamide, ifosphamide, actinomycin-d and N-(4-hydroxyphenyl) retinamide (4-HPR). Examples of angiogenesis inhibitors include but are not limited to 2-methoxyestradiol (2-ME), AG3340, Angiostatin, antithrombin-III, Anti-VEGF antibody, Batimastat, bevacizumab (Avastin), BMS-275291, CA1, Canstatin, combretastatin, Combretastatin-A4 phosphate, CC-5013, captopril, celecoxib, Dalteparin, EMD121974, Endostatin, Erlotinib, Gefitinib, Genistein, Halofuginone, ID 1, ID3, IM862, Imatinib mesylate, Inducible protein-10, Interferon-alpha, Interleukin-12, Lavendustin-a, LY317615, or AE-941, Marimastat, Mapsin, Medroxyprogesterone acetate, Meth-1, Meth-2, Neovastat, Osteopontin cleaved product, PEX, Pigment epithelium growth factor (PEGF), platelet growth factor 4, prolactin fragment, proliferin-related protein (PRP), PTK787/ZK222584, recombinant human platelet factor-4(rPF4), restin, squalamine, SU5416, SU6668, Suramin, Taxol, Tecogalan, Thalidomide, Tetrathiomolybdate (TM), Thrombospondin, TNP-470, Troponin I, Vasostatin, VEGF1, VEGF-TPvAP and ZD6474. In some embodiment the angiogenesis inhibitor is a VRGF antagonist. The VEGF antagonist may be a VEGF binding molecule. VEGF binding molecule include VEGF antibodies, or antigen binding fragment (s) thereof. One example of a VEGF antagonist is NeXstar.

Examples of categories of chemotherapeutic agents that may be used in any of the methods or agents disclosed herein include, but are not limited to, DNA damaging agents and these include topoisomerase inhibitors (e.g., etoposide, camptothecin, topotecan, irinotecan, teniposide, mitoxantrone), anti-microtubule agents (e.g., vincristine, vinblastine), antimetabolite agents (e.g., cytarabine, methotrexate, hydroxyurea, 5-fluorouracil, flouridine, 6-thioguanine, 6-mercaptompurine, fludarabine, pentostatin, chlorodeoxyadenosine), DNA alkylating agents (e.g., cisplatin, mecholorethamine, cyclophosphamide, ifosphamide, melphalan, chlorambucil, busulfan, thiotepa, carmustine, lomustine, carboplatin, dacarbazine, procarbazine) and DNA strand break inducing agents (e.g., bleomycin, doxorubicin, daunorubicin, idarubicin, mitomycin C).

Chemotherapeutic agents include synthetic, semisynthetic and naturally derived agents. Important chemotherapeutic agents include, but are not limited to, Avicine, Aclarubicin, Acodazole, Acronine, Adozelesin, Adriamycin, aldesleukin, Alitretinoin, AUopurinol sodium, Altretamine, Ambomycin, Ametantrone acetate, Aminoglutethimide, Amsacrine, Anastrazole, Annonaceous Acetogenins, Anthramycin, Asimicin, Asparaginase, asperlin, Azacitidine, azetepa, Azotomycin, batimastat, benzodepa, bexarotene, Bicalutamide, Bisantrene, Bisnafide, Bizelesin, Bleomycin, Brequinar, Bropirimine, Bullatacin, Busulfan, Cabergoline, cactinomycin, calusterone, caracemide, carbetimer, carboplatin, carmustine, carubicin, carzelesin, cedefingol, chlorambucil, celecoxib, cirolemycin, cisplatin, cladribine, crisnatol, cyclophosphamide, cytarabine, dacarbazine, DACA, dactinomycin, Daunorubicin, daunomycin, Decitabine, denileukin, Dexormaplatin, Dezaguanine, Diaziquone, Docetaxel, Doxorubicin, Droloxifene, Dromostalone, Duazomycin, Edatrexate, Eflornithine, Elsamitrucin, Estramustine, Etanidazole, Etoposide, Etoprine, Fadrozole, Fazarabine, Fenretinide, Floxuridine, Fludarabine, Fluorouracil, Flurocitabine, 5-FdUMP, Fosquidone, Fosteuecine, FK-317, FK-973, FR-66979, FR-900482, Gemcitabine, Gemtuzumab, Ozogamicin, Gold Aul 98, Goserelin, Guanacone, Hydroxyurea, Idarubicin, Ilmofosine, Interferon alpha and analogs, Iproplatin, irinotecan, Lanreotide, Letrozole, Leuprolide, Liarozole, Lometrexol, Lomustine, Losoxantrone, masoprocol, Maytansine, Mechlorethamine, Megestrol, Melengestrol, Melphalan, Menogaril, Metoprine, maturedepa, mitindomide, Mitocarcin, Mitogillin, Mitomalacin, Mitomycin, Mitomycin C, Mitosper, Mitotane, Mitoxantrone, Mycophenolic acid, Nocodazole, Nogalamycin, Oprelvekin, ormaplatin, Oxisuran, Paclitaxel, pamidronate, pegaspargase, Peliomycin, Pentamustine, Peplomycin, Perfosfamide, Pipobroman, Piposulfan, Piroxantrone, Plicamycin, Plomestane, Porfimer, Porfiromycin, Prednimustine, procarbazine, Puromycin, Pyrazofurin, Riboprine, Rituximab, Rogletimide, Rolliniastatin, safingol, Samarium, Semustine, Simtrazene, Sparfosate, Sparsomycin, spirogermanium, Spiromustine, Spiroplatin, Squamocin, Squamotacin, streptonigrin, streptozocin, SrC12, Sulphofenur, Talisomycin, Taxane, Toxoid, Tecoglan, Tegafur, teloxantrone, Temoporfin, teniposide, Teroxirone, Testolactone, Thiamiprine, Thiotepa, Thymitaq, Tiazofurin, Tirapazamine, Tomudex, Top-53, Topotecan, Toremixifme, Trastuzumab, Trestolone, triciribine, Triciribine, Trimetrexate, trimetrexate glucuronate, Triptorelin, Tubulozole, uracil mustard, Uredepa, valrubicin, vapreotide, Vinblastine, Vincristine, Vindesine, Vinepidine, Vinglycinate, Vinleurosine, Vinorelbine, Vinrosidine, Vinzolidine, Vorozole, Zeniplatin, Zinostatin, Zorubicin, 2-cholrodeoxyrubicine, 2′-deoxyformycin, 9-aminocamptothecin, raltitrexed, N-propargyl-5,8-didezafolic acid, 2-cholo-2′arabinofluoro-2′ deoxyadenosine, 2-cholo-2′-deoxyadenosine, anisomycin, Trichostatin, hPRL-G129R, CEP-751, Linomide, Sulfur mustard, nitrogen mustard, N-methyl-N-nitrosourea, fotemustine, Streptozotocin, dacarbazine, mitozolomide, temozolomide, AZQ, ormaplatin, CI-973, DWA21 14R, JM216, JM335, Bisplatinum, Tomudex, azacitidine, cytrabincine, gemcitabine, 6-mercaptopurine, Hypoxanthine, Teniposide, CPT-11, Doxorubicin, Daunorubicin, Epirubicin, darubicin, losoxantrone, amsacrine, pyrazoloacridine, all trans retinol, 14-hydroxy-retro-retinol, all-trans retinoic acid, N-(4-hydroxyphenyl) retinamide, 13-cisretinoic acid, 3-methyl TTNEB, 9-cisretenoic acid, fludarabine, and 2-Cda.

Other chemotherapeutic agent include: 20-epi1,25-dihydroxyvitamin-D3, 5-ethynyl uracil, abiraterone, aclarubicin, acylfulvene, adecylpenol, adozelesin, aldesleukin, ALL-TK antagonists, altretamine, ambumastine, amidox, amifostine, amino levulinic acid, anagrelide, anastrozole, andrographolide, angiogenesis inhibitors, antagonist D, antagonists D, antarelix, anti-dorsalizing morphogenetic protein-1, antiandrogen, antiestrogen, antineoplastone, antisense oligonucleotides, aphidicolin, apoptosis gene modulators, apoptosis regulators, apurinic acid, ara-cdp-dl-PTBA, arginine aminase, asulacrine, atamestine, atrimustine, axinamastine 1 and axinamastine 2, axinamastine 3, azasetron, azatoxin, azatyrosine, baccatin III derivatives, balanol, BCR/ABL antagonist, benzochlorins, benzoylsaurosporine, beta lactam derivatives, beta-alethine, perillyl alcohol, phenozenomyein, phenyl acetate, phosphatase inhibitors, picibanil, pilocarbine and salts or analogs thereof, pirarubucin, piritrexim, placetin A, placetin B, plasminogen activator inhibitor, platinum complex, phenyl ethyl isothiocyanate and analogs thereof, platinum compounds, platinum triamine complex, podophylotoxin, porfimer sodium, porphyromycin, propyl bis acridones, prostaglnadins J2, protease inhibitors, protein A based immune modulators, PKC inhibitors, microalgal, protein tyrosine phosphatase inhibitors, purine nucleoside phosphory lase inhibitors, purpurins, pyrazoloacridines, pyridoxylated hemoglobin polyoxyethylene conjugate, raf antagonists, raltitrexed, ramosetron, ras farnesyl protein tranferase inhibitors, rasinhibitors, ras-GAP inhibitors, ratellitptine demethylated, Rhenium Re 186 etidronate, rhizoxine, ribozyme, RII retinide, rogletimide, rosagliatazone and analogs and derivatives thereof, rohitukine, romurtide, roquinimex, rubiginone BI, ruboxyl, safingol, saintopin, SarCNU, sarcophytol A, sargrmostim, sdi 1 mimetics, semustine, senescence derived inhibitor 1, sense oligonucleotide, signal transduction inhibitors, signal transduction modulators, single chain antigen binding protein, sizofiran, sobuzoxane, sodium borocaptate, sodium phenyl acetate, solverol, somatomedin binding protein, sonermin, sparfosic acid, spicamycin D, spiromustin, splenopentine, spongistatin 1, squalamine, stem cell inhibitor, stem cell division inhibitor, stipiamide, stromelysin, sulfinosine, superactive vasoactive intestinal peptide antagonists, suradista, siramin, swainsonine, synthetic glycosaminoglycans, tallimustine, tamoxifen methiodide, tauromustine, tazarotene, tacogalan sodium, tegafur, tellurapyrilium, telomerase inhibitors, temoporfin, tmeozolomide, teniposide, tetrachlorodecaoxide, tetrazomine, thaliblastine, thalidomide, thiocoraline, thrombopoetin and mimetics thereof, thymalfasin, thymopoetin receptor agonist, thymotrinan, thyroid stimulating harmone, tin ethyl etiopurpin, tirapazamine, titanocene and salts thereof, topotecan, topsentin, toremifene, totipotent stem cell factors, translation inhibitors, tretinoin, triacetyluridine, tricribine, trimetrexate, triptorelin, tropisetron, turosteride, tyrosine kinase inhibitors, tyrphostins, UBC inhibitors, ubenimex, urogenital sinus derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozol, zanoterone, zeniplatin, zilascorb and zinostatin. Other chemotherapeutic agents include: antiproliferative agents (e.g., piritrexim isothiocyanate), antiprostatic hypertrophy agents (sitogluside), Benign prostatic hyperplasia therapy agents (e.g., tomsulosine, RBX2258), prostate growth inhibitory agents (pentomone) and radioactive agents: Fibrinogen 1125, Insulin 1125, Iobenguane 1123, Iodipamide sodium 1131, lodoantipyrine 1131, Iodocholesterol 1131, Iodopyracet 1125, Iofetamine HCL 1123, Iomethin 1131, Iomethin 1131, Iothalamate sodium 1125, Iothalamate 1131, Iotyrosine 1131, Liothyronine 1125, Merosproprol Hgl 97, Methyl ioodobenzo guanine (MIBG-1131 or MIBGI 123) selenomethionine Se75, Technetium Tc99m furifosmin, technetium Tc99m gluceptate, Tc99m Biscisate, Tc99m disofenin, TC99m gluceptate, Tc99m lidofenin, Tc99m mebrofenin, Tc99m medronate and sodium salts thereof, Tc99m mertiatide, Tc99m oxidronate, Tc99m pentetate and salts thereof, Tc99m sestambi, Tc99m siboroxime, Tc99m succimer, Tc99m sulfur colloid, Tc 99m teboroxime. Tc 99m Tetrofosmin, Tc99m Tiatide, Thyroxine 1125, Thyroxine 1131, Tolpovidone 1131, Triolein 1125 and Treoline 1125, and Treoline 131, MIBG-1123 and MIBG 1131 are especially preferred chemotherapeutic agents for co-administration with the nitrofuran containing pharmaceutical composition of invention.

Another category of chemotherapeutic agents are anticancer supplementary potentiating agents, e.g., antidepressant drugs (Imipramine, desipramine, amitriptyline, clomipramine, trimipramine, doxepin, nortriptyline, protriptyline, amoxapine, and maprotiline), or no-trycyclic anti-depressant drugs (sertraline, trazodone and citalopram), Ca++ antagonists (verapamil, nifedipine, nitrendipine and caroverine), calmodulin inhibitors (prenylamine, trifluoperazine and clomipramine), Amphotericin B, Triparanol analogs (e.g., Tamoxifen), antiarrhythmic drugs (e.g., quinidine), antihypertensive drugs (e.g., reserpine), thiol depleters (e.g., buthionine and sulfoximine) and multiple drug resistance reducing agents such as Cremophor EL.

In some embodiments, the composition or biodegradable agent further comprises a diagnostic agent. Such embodiments are not limited to a particular type of diagnostic agent.

In some embodiments, the diagnostic agent is an antibody. In some embodiments, the antibody is 3F8, Abagovomab, Abciximab, Abituzumab, Abrilumab, Acritumomab, Actoxumab, Adalimumab, Adalimumab-atto, Adecatumumab, Ado-trastuzumab emtansine, Aducanumab, Afasevikumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, Crenezumab, Crotedumab, CR6261, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Inebilizumab, Infliximab, Infliximab-dyyb, Intetumumab, Inolimomab, Inotuzumab ozogamicin, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab emtansine, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Mapatumumab, Margetuximab, Maslimomab, Mavrilimumab, Matuzumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tildrakizumab, Tigatuzumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, or Zolimomab aritox.

In some embodiments, the diagnostic agent is Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase Clostridium histolyticum, Darbepoetin alfa, Denileukin diftitox, Dornase alfa, Dulaglutide, Ecallantide, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1 a, Interferon beta-1 b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a, Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, or Ziv-aflibercept.

In some embodiments, the diagnostic agent is tuberculin purified protein derivative, hyrotropin alpha, secretin, soluble transferrin receptor, troponin, B-type natriuretic peptide, iobenguane I 123, florbetapir F 18, perflutren, gadoterate meglumine, florbetaben F 18, flutemetamol F 18, gadoterate meglumine, isosulfan blue, regadenoson, technetium Tc 99m tilmanocept, florbetaben F 18, perflutren, regadenoson, or flutemetamol F 18.

In some embodiments, the composition or biodegradable agent further comprises an agent capable of relaxing (e.g., opening, dilating) the hepatopancreatic anatomical region (e.g., hepatopancreatic ampulla, hepatopancreatic duct, Ampulla of Vater, Sphincter of Oddi) in a subject. Such embodiments are not limited to a particular type of agent capable of relaxing (e.g., opening, dilating) the hepatopancreatic anatomical region (e.g., hepatopancreatic ampulla, hepatopancreatic duct, Ampulla of Vater, Sphincter of Oddi) in a subject. In some embodiments, the agent capable of relaxing (e.g., opening, dilating) the hepatopancreatic anatomical region is selected from nitroglycerin, scopolamine butylbromide, hymecromone (7-hydroxy-4-methylumbelliferone), botulinum toxin, vasoactive intestinal polypeptide, cholecystokinin-8, and secretin. In certain embodiments, the present invention provides methods for treating, preventing, and/or ameliorating the symptoms of a condition in a subject, the method comprising administering to a subject a pharmaceutically effective amount of a composition comprising biodegradable agent associated with (e.g., complexed, conjugated, encapsulated, absorbed, adsorbed, admixed) a therapeutic agent.

In some embodiments, the composition or biodegradable agent further comprises a diagnostic agent and an agent capable of relaxing (e.g., opening, dilating) the hepatopancreatic anatomical region (e.g., hepatopancreatic ampulla, hepatopancreatic duct, Ampulla of Vater, Sphincter of Oddi) in a subject.

In certain embodiments, the present invention provides methods for treating, preventing, ameliorating the symptoms of, and/or diagnosing a condition in a subject, the method comprising administering to a subject a pharmaceutically effective amount of a composition comprising biodegradable agent associated with (e.g., complexed, conjugated, encapsulated, absorbed, adsorbed, admixed) one or more of a therapeutic agent, a diagnostic agent, and an agent capable of relaxing (e.g., opening, dilating) the hepatopancreatic anatomical region (e.g., hepatopancreatic ampulla, hepatopancreatic duct, Ampulla of Vater, Sphincter of Oddi).

In some embodiments, the composition is co-administered (e.g., prior to, simultaneously, or following) with an agent capable of relaxing (e.g., opening, dilating) the hepatopancreatic anatomical region (e.g., hepatopancreatic ampulla, hepatopancreatic duct, Ampulla of Vater, Sphincter of Oddi) in the subject. Such methods are not limited to specific agents capable of relaxing (e.g., opening, dilating) the hepatopancreatic anatomical region (e.g., hepatopancreatic ampulla, hepatopancreatic duct, Ampulla of Vater, Sphincter of Oddi). In some embodiments, the agent capable of relaxing (e.g., opening) the hepatopancreatic anatomical region (e.g., hepatopancreatic ampulla, hepatopancreatic duct, Ampulla of Vater, Sphincter of Oddi) is selected from nitroglycerin, scopolamine butylbromide, hymecromone (7-hydroxy-4-methylumbelliferone), botulinum toxin, vasoactive intestinal polypeptide, cholecystokinin-8, and secretin.

Such methods are not limited to a particular type of subject. In some embodiments, the subject is a mammalian subject. In some embodiments, the subject is a human subject.

Such methods are not limited to treating, preventing, and/or ameliorating the symptoms of a particular condition.

In some embodiments, the condition is any disease related to the pancreas (e.g., any type of pancreatic cancer, pancreatitis, diabetes). In some embodiments, the condition is any disease related to the bile duct (e.g., cholangitis, bile duct leaks, biliary stricture, biliary stones, bile duct cancer (e.g., cholangiocarcinoma). In some embodiments, the condition is any disease related to the gall bladder (e.g., gall stones, gall bladder carcinoma). In some embodiments, the condition is any disease related to the liver (e.g., hepatitis, cholangitis, biliary atresia, alpha-1 antitrypsin deficiency, alagille syndrome, progressive familial intrahepatic cholestasis, Langerhans cell histiocytosis, hepatic hemangioma, polycystic liver disease, liver cancer).

In some embodiments, such methods further comprise administering other therapies such as, for example, radiation therapy, surgery, conventional chemotherapy or with a combination of one or more additional therapies. Such other active ingredient includes, but is not limited to glutathione antagonists, angiogenesis inhibitors, chemotherapeutic agent(s) and antibodies (e.g., cancer antibodies). The agents described in this invention may be administered simultaneously or sequentially. The separation in time between administrations may be minutes, hours, days or it may be longer.

Such methods are not limited to a particular manner of administration of the composition comprising biodegradable agent associated with (e.g., complexed, conjugated, encapsulated, absorbed, adsorbed, admixed) one or more of a therapeutic agent, a diagnostic agent, and an agent capable of relaxing (e.g., opening, dilating) the hepatopancreatic anatomical region (e.g., hepatopancreatic ampulla, hepatopancreatic duct, Ampulla of Vater, Sphincter of Oddi). In some embodiments, the composition is administered via transpapillary administration.

In some embodiments, the composition is administered orally. In some embodiments, the composition is administered by oral gavage. In some embodiments, the composition is administered intratumorally. In some embodiments, the composition is administered intravenously. In some embodiments, the composition is administered subcutaneously. However, administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising a comprising biodegradable agents (e.g., microparticles, nanoparticles) associated with (e.g., complexed, conjugated, encapsulated, absorbed, adsorbed, admixed) a) one or more of a therapeutic agent, diagnostic agent, and an agent capable of relaxing (e.g., opening, dilating) the hepatopancreatic anatomical region (e.g., hepatopancreatic ampulla, hepatopancreatic duct, Ampulla of Vater, Sphincter of Oddi), and b) one or more of an agent capable of relaxing (e.g., opening, dilating) the hepatopancreatic anatomical region (e.g., hepatopancreatic ampulla, hepatopancreatic duct, Ampulla of Vater, Sphincter of Oddi) in a subject, and additional therapeutic agents capable of treating, preventing and/or ameliorating the symptoms of a disease or condition related to the pancreatic/biliary anatomical region.

### EXPERIMENTAL

The following examples are provided to demonstrate and further illustrate certain preferred embodiments of the present invention and are not to be construed as limiting the scope thereof. As used herein, the use of personal pronouns such as “we”, “I”, and/or “our” refers to the inventors.

### Example I

### Methods

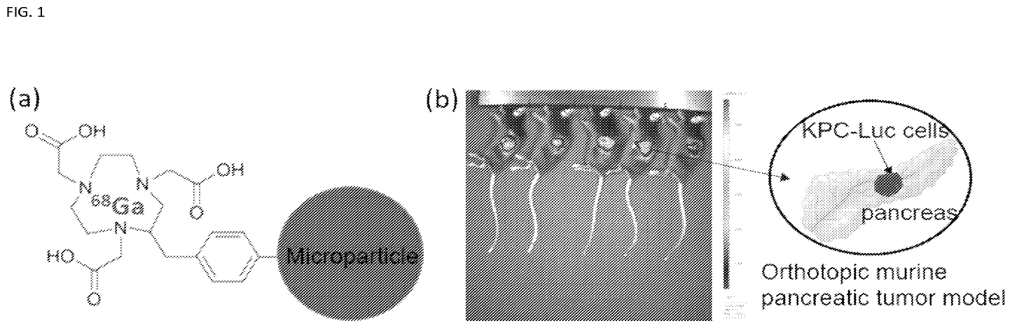

Polystyrene microparticles (from 1.0-8.0 μm in diameter, PSMPs) and chelator NOTA (2-S-(4-isothiocyanatobenzyl)-1,4,7-triazacyclononane-1,4,7-triacetic acid) were purchased from Fisher Scientific. Iron-coated-PSMPs (Fe-PSMPs, average size: 3.0 μm) were purchased from Spherotech Inc., and mineral supplement Nano-calcium was purchased from AceNano Inc. SiO₂ nanoparticles (NPs) were synthesized as reported in a previously published paper (ACS nano, 2019, 13, 12148). Radioisotope ⁶⁸Ga (t_{1/2}=68 min) was produced at the University of Michigan, Ann Arbor. ⁶⁸Ga was labeled on the surface of PSMPs through chelator NOTA as shown in FIG. 1a.

All animal experiments were in accordance with the approval of the Institutional Animal Care and Use Committee (IACUC) at the University of Michigan, Ann Arbor. Female C57BL/6 mice 6-8 weeks of age (Jackson Laboratories) were orthotopically inoculated in pancreas with KPC-Luc cells (1.5×10⁴). 14 days after tumor inoculation, the growth of orthotopic tumor were visualized by imaging bioluminescence of KPC-Luc cell as shown in FIG. 1b. During the biodistribution studies, ⁶⁸Ga-PSMPs, Fe-PSMPs or Nano-Calcium were administered in mice via oral gavage. Mice were euthanized 2 hours after oral gavage. The pancreas and other major organs were collected and wet-weighted and the radioactivity in the organs were measured by a gamma counter (Wizard 2 1-Detector, PerkinElmer).

### Results

### Oral Administration of Microparticles Leads to Efficient Accumulation in Pancreas.

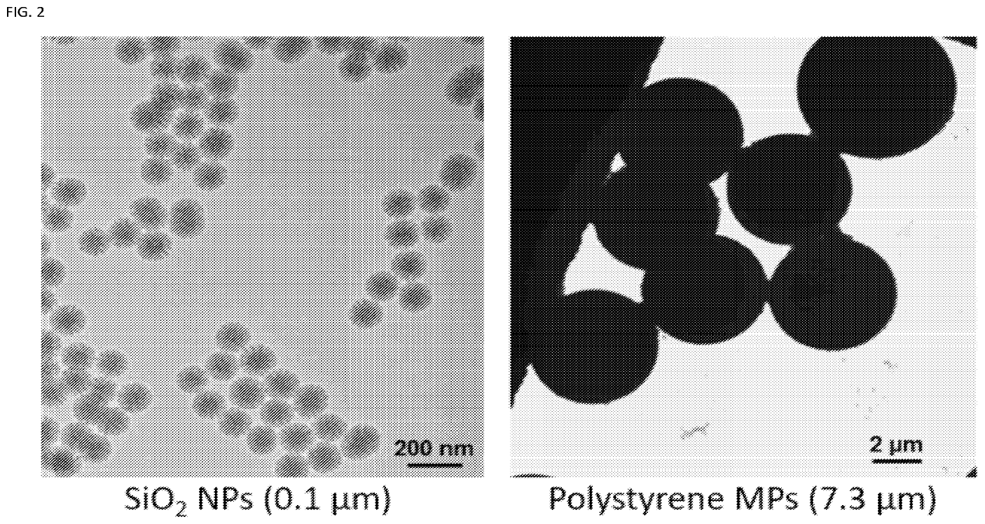

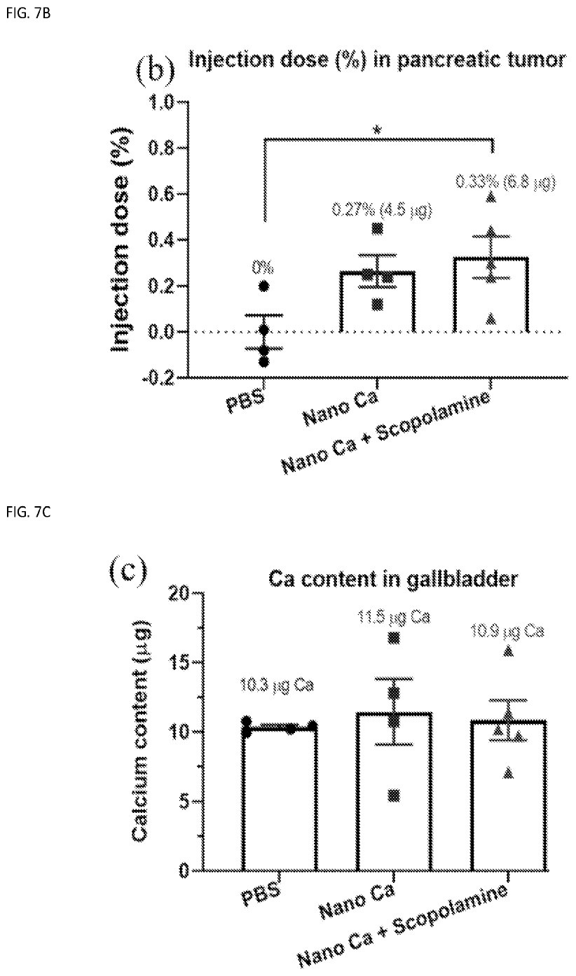

We (the inventors) examined accumulation of microparticles (a. PSMPs, 7.3 μm in diameter; b. Fe-PSMPs, 3.0 μm in diameter; c. Nano-calcium MPs, 0.5-5.0 μm in diameter) or nanoparticles (NPs, 100 nm om diameter) in pancreas and other organs after oral administration into mice bearing orthotopic pancreatic tumor. Four representative electron microscope (TEM or SEM) images for MPs and NPs are shown in FIGS. 2, 6a, and 7a.

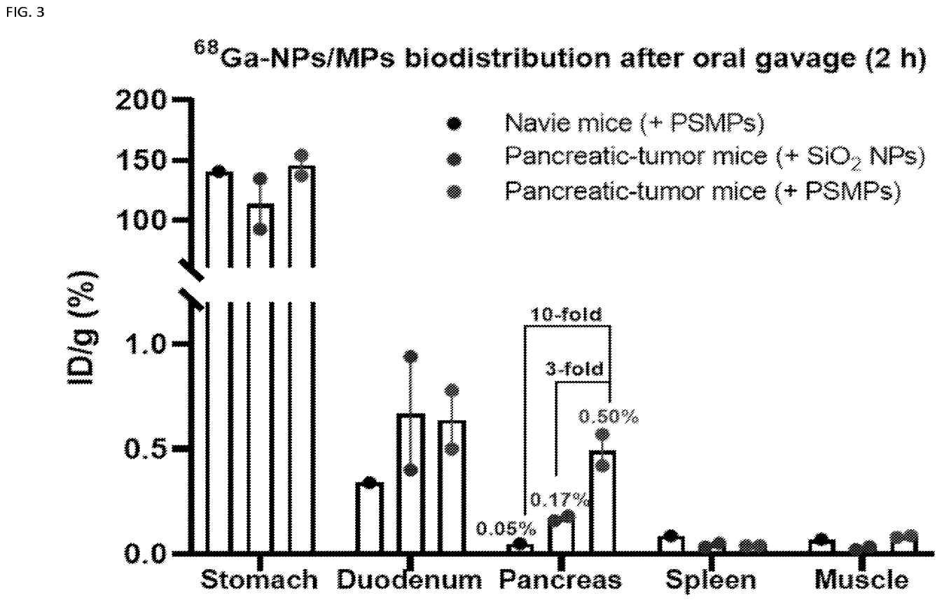

After labeling with radioisotope ⁶⁸Ga, SiO₂ NPs or polystyrene MPs were administered to naive mice or pancreatic tumor-bearing mice by oral gavage. Separately, Fe-PSMPs or Nano-calcium were directly administered to pancreatic tumor-bearing mice by oral gavage. After 2 hrs, various organs were harvested and analyzed for the biodistribution of NPs or MPs (FIG. 3-7).

In healthy, naïve mice, the pancreatic distribution of polystyrene MPs was minimal. This is as we expected because the Sphincter of Oddi limits the influx of fluid or food particles from duodenum to pancreas in healthy condition. However, in pancreatic tumor-bearing mice, we observed increased accumulation of both SiO₂ NPs and polystyrene MPs in pancreas. Importantly, polystyrene MPs with an average size of 7.3 μm showed 3-fold higher accumulation in pancreas, compared with SiO₂ NPs with an average size of 100 nm. Polystyrene MPs exhibited 10-fold higher pancreatic accumulation in pancreatic tumor-bearing mice, compared naïve mice. This indicates that the presence of tumor cells in pancreas disrupts the functions of Sphincter of Oddi, thus allowing large MPs to enter the pancreas through the pancreatic duct.

### Sphincter-Relaxation Drugs can Further Improve Accumulation of MPs in Pancreas.

The Sphincter of Oddi control the efflux of pancreatic juice from pancreas to duodenum while also limiting the influx of intestinal fluid into pancreas. Hence, we hypothesized that relaxing the Sphincter of Oddi via drug treatments would allow the increased influx of microparticles from duodenum into the pancreas.

As indicated above, scopolamine butylbromide is an FDA-approved anticholinergic drug that exerts a smooth-muscle relaxing/spasmolytic effect.

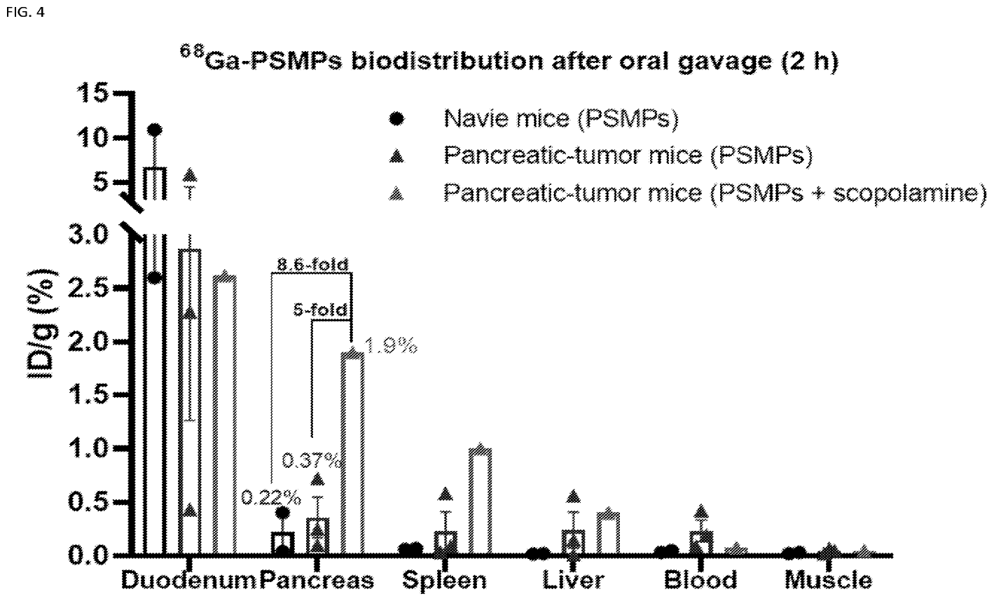

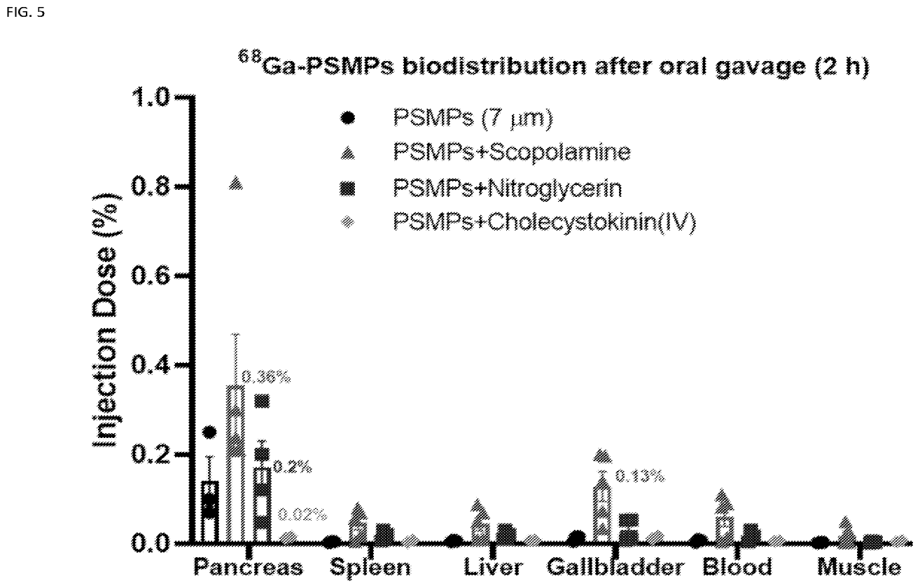

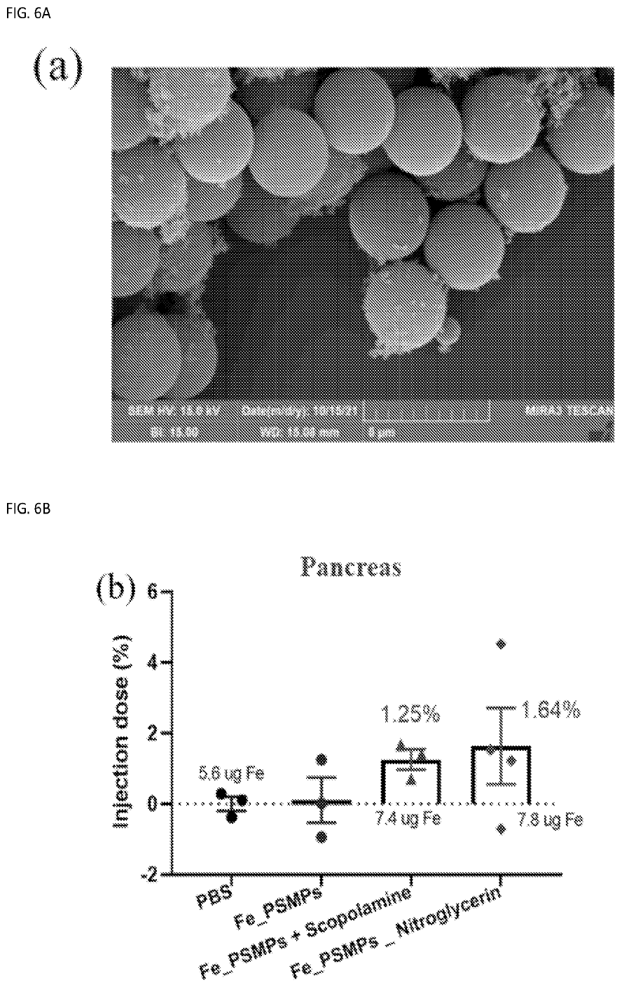

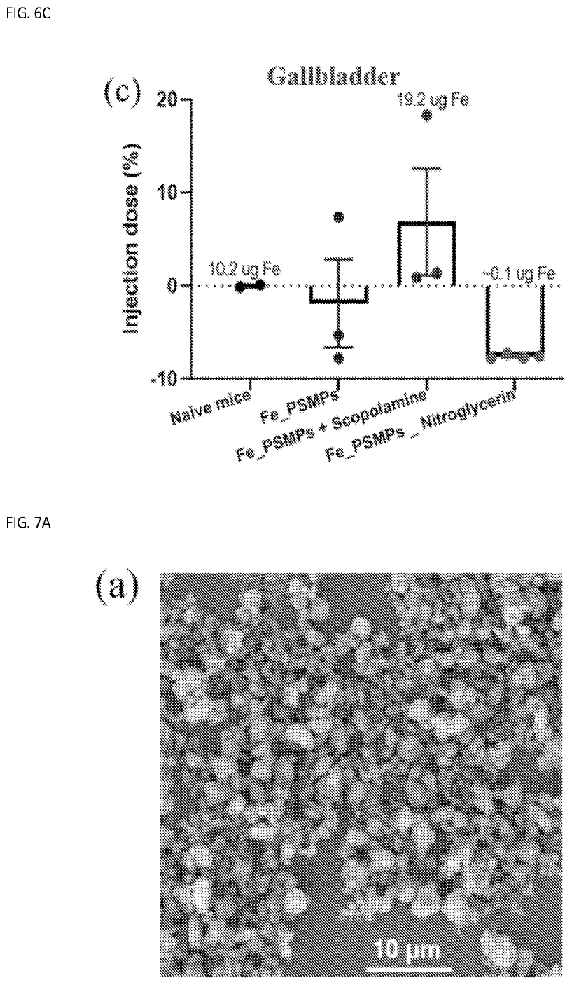

We tested co-administration of scopolamine butylbromide (5 mg/kg) or nitroglycerin with polystyrene MPs or Nano-calcium MPs via oral gavage in pancreatic tumor-bearing mice (FIGS. 4-7 and 5). Addition of scopolamine butylbromide led to a 3-5-fold increase in the accumulation of MPs in the pancreas (FIGS. 4 and 5). Addition of nitroglycerin led to a 10 fold increase in the accumulation of Fe-PSMPs in the pancreas (FIG. 6a). We observed 1.9% injected MP dose per gram of pancreas, which is equivalent to 0.4% of injected MPs in the pancreas. Similarly, we observed 1.3% of injection Fe-PSMPs and 0.3% of injected Nano-calcium MPs in pancreas. In addition, we observed 0.1%-6% injected MPs accumulated in gallbladder (FIGS. 5, 6c and 7c). While this value is slightly lower than the reported value of 0.7% injected drug dose in tumor tissues after IV administration (Nature reviews materials, 2016, 1, 1-12), our oral formulation does not lead to systemic drug exposure; thus, we anticipate much-reduced toxicity. Furthermore, FIGS. 4 and 5 show that MPs orally administered with scopolamine butylbromide or nitroglycerin also accumulated in spleen and liver/gallbladder, indicating the use of oral MP formulation for targeting these organs.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition comprising one or more biodegradable agents (e.g., microparticles or a nanoparticles) associated with (e.g., complexed, conjugated, encapsulated, absorbed, adsorbed, admixed) one or more therapeutic agents (e.g., agents capable of treating and/or ameliorating the symptoms of conditions associated with the pancreas (e.g., pancreatic cancer, pancreatitis, diabetes), conditions associated with the bile duct (e.g., cancer associated with the bile duct), conditions associated with the liver (e.g., liver cancer), and/or conditions associated with the gall bladder (e.g., gall bladder cancer)).

2. The composition of claim 1, wherein the size of the diameter of the biodegradable agent is between approximately 0.1 to 100 μm.

3. The composition of claim 1, wherein the size of the diameter of the biodegradable agent is between approximately 5 to 20 μm.

4. The composition of claim 1, wherein the biodegradable agent is a microparticle or nanoparticle.

5. The composition of claim 3, wherein the microparticle is selected from corn zein, n-acetylglucosamine, cyclo-dextrin, polysaccharides copolymers of lactic acid and glycolic acid, starch-borate complexes, polyvinyl alcohol-borate complexes, polypeptides, polyvinyl alcohol, gelled protein solution, soluble potato starch hydrolysate, alginate, cross-linked alginate, starches, dextrans, celluloses, hydroxy-propyl cellulose, chitin, de-acetylated chitin, chitosan, hyaluronic acid and its derivatives, collagen, albumin, gelatin, glycosaminoglycans, polylactic acid (PLA), copolymers of lactic and aminocaproic acid, polyglycolic acid (PGA), poly(lactic-co-glycolic acid) (PLGA), polyoxyethylene-polyoxypropylene block copolymers, poly(DL-lactamide), copolymers of leucine and glutamic acid, polystyrene, polyesters, poly(ortho esters), non-biodegradable polyurethanes, polyureas, poly(vinyl alcohol), polyanhydrides, polyamides, poly (tetrafluoroethylene), poly(ethylene vinyl acetate), polypropylene, polyacrylate, non-biodegradable polycyanoacrylates, polyacryl starch, non-biodegradable polyurethanes, polymethacrylate, poly(methyl methacrylate), polyethylene, polypyrrole, polyanilines, polythiophene, and poly(ethylene oxide).

6. The composition of claim 3, wherein the nanoparticle is between 5 to 500 nm.

7. The composition of claim 3, wherein the nanoparticle is selected from the group consisting of albumin-based nanoparticles (e.g., bovine serum albumin, human serum albumin), silica nanoparticles, gold nanoparticles, graphene nanoparticles, graphene oxide nanoparticles, corn zein nanoparticles, polystyrene nanoparticles, fullerenes, endohedral metallofullerenes buckyballs, trimetallic nitride templated endohedral metallofullerenes, single-walled and mutli-walled carbon nanotubes, branched and dendritic carbon nanotubes, gold nanorods, silver nanorods, single-walled and multi-walled boron/nitrate nanotubes, carbon nanotube peapods, carbon nanohorns, carbon nanohorn peapods, liposomes, nanoshells, dendrimers, any nanostructures, microstructures, or their derivatives formed using layer-by-layer processes, self-assembly processes, or polyelectrolytes, microparticles, quantum dots, superparamagnetic nanoparticles, nanorods, cellulose nanoparticles, glass and polymer micro- and nano-spheres, biodegradable PLGA micro- and nano-spheres, gold nanoparticles, silver nanoparticles, carbon nanoparticles, iron nanoparticles, a modified micelle, metal-polyhistidine-DOPE@liposome, metal-polyhistidine-PEG, 4arm-PEG-polyhistidine-metal hydrogels, and sHDL-polyhistidine, and metal-organic framework (MOF) coordination polymer (CP).

8. The composition of claim 1, wherein the biodegradable agent is a fiber containing prebiotic agent.

9. The composition of claim 8, wherein the fiber containing prebiotic agent is selected from epigallocatechin gallate (EGCG), fucoidan, potato starch, oligofructose and inulin.

10. The composition of claim 9,
wherein the fiber containing prebiotic agent is a gel-based or particle-based inulin formulation having an average degree of polymerization at or higher than 20 and at or less than 47 (e.g., approximately 28 (e.g., 23, 24, 25, 26, 27, 28, 29, 30, 31, 32, 33)), and/or wherein the gel-based inulin formulation comprises one or more prebiotic compounds selected from a fructo-oligosaccharide, a short-chain fructo-oligosaccharide, an isomalt-oligosaccharide, a transgalacto-oligosaccharide, a pectin, a xylo-oligosaccharide, a chitosan-oligosaccharide, a beta-glucan, an arable gum modified starch, a resistant potato starch, guar gum, bean gum, gelatin, glycerol, a polydextrose, a D-tagatose, an acacia fiber, carob, an oat, and a citrus fiber.

11. The composition of claim 1, wherein the therapeutic agent is an immune checkpoint inhibitor (ICI).

12. The composition of claim 11, wherein the ICI is capable of binding to, blocking, and/or inhibit the activity of one or more of CTLA-4, PDL1, PDL2, PD1, BTLA, HVEM, TIM3, GAL9, LAG3, VISTA, KIR, 2B4, CD160 and CGEN-15049.

13. The composition of claim 11, wherein the ICI is selected from Tremelimumab (CTLA-4 blocking antibody), anti-OX40, PD-L1 monoclonal Antibody (Anti-B7-H1: MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

14. The composition of claim 1, wherein the therapeutic agent is an immunostimulatory agent.

15. The composition of claim 14, wherein the immunostimulatory agent is selected from anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, inhibitors of IDO, CpG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2, IFN-a, Flt-3L), IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MEDI9197, glucopyranosyl lipid adjuvant (GLA), GLA-SE, CD1d ligands (such as C20:2, OCH, AH04-2, a-galatosylceramide, α-C-galatosylceramide, α-mannosylceramide, α-fructosylceramide, β-galatosylceramide, β-mannosylceramide), STING agonists (e.g. cyclic dinucleotides, including Cyclic [G(3′,5′)pA(3′,5′)p], Cyclic [G(2′,5′)pA(3′,5″)p], Cyclic [G(2′,5″)pA(2′,5′)p], Cyclic diadenylate monophosphate, Cyclic diguanylate monophosphate), CL401, CL413, CL429, Flagellin, RC529, E6020, imidazoquinoline-based small molecule TLR-7/8a (including its lipidated analogues), virosomes, AS01, AS02, AS03, AS04, AS15, IC31, CAF01, ISCOM, Cytokines (such as GM-CSF, IL-2, IFN-a, Flt-3L), bacterial toxins (such as CT, and LT), cations (such as Zn²⁺, Mn²⁺, Ca²⁺, Fe²⁺, Fe³⁺, Cu²⁺, Ni²⁺, Co²⁺, Pb²⁺, Sn²⁺, Ru²⁺, Au²⁺, Mg²⁺, VO²⁺, Al³⁺, Co³⁺, Cr³⁺, Ga³⁺, Tl³⁺, Ln³⁺, MoO³⁺, Cu⁺, Au⁺, Tl⁺, Ag⁺, Hg²⁺, Pt²⁺, Pb²⁺, Hg²⁺, Cd²⁺, Pd²⁺, Pt⁴⁺, Na⁺, K⁺, and relative phosphate or carbonate salt), any derivative of an immunostimulatory agent, and any combination of immunostimulatory agents.

16. The composition of claim 15, wherein the one or more STING agonists is selected from the group consisting of cGAMP, cdiAMP, cdiGMP, cAIMP, 2′3′-cGAMP, 3′3′-cGAMP, c-di-AMP, c-di-GMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, 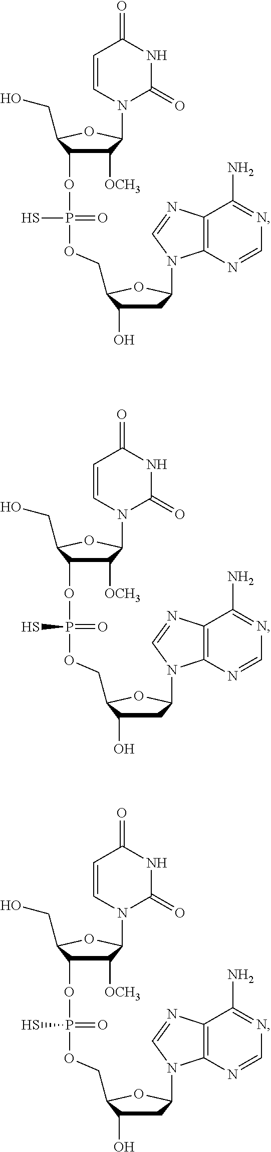 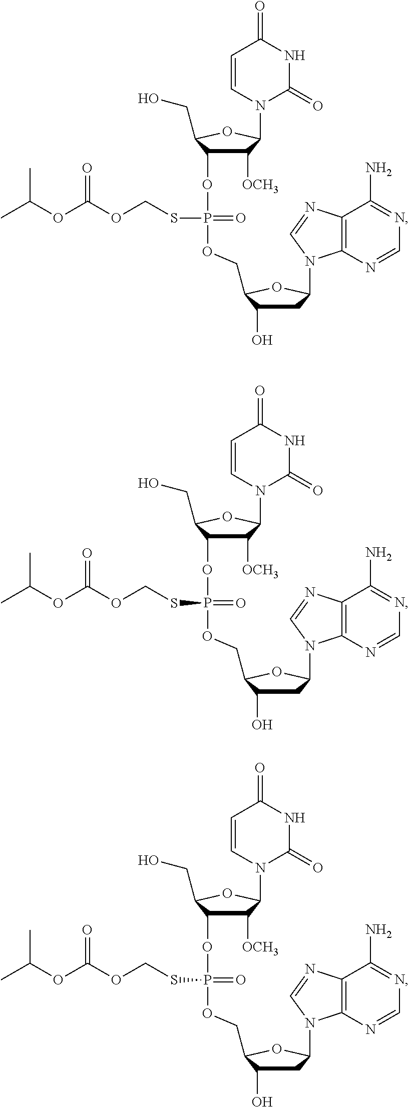
SB11285 (Spring Bank Pharmaceuticals), Gemcitabine ( 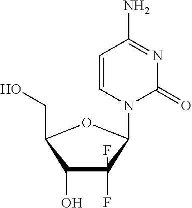
STING-agonist-C11 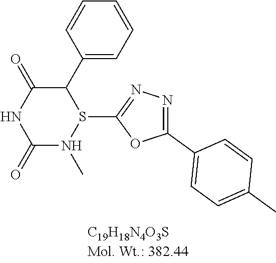
STING agonist-1 ( 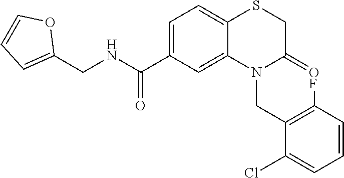
STING agonist G10 ( 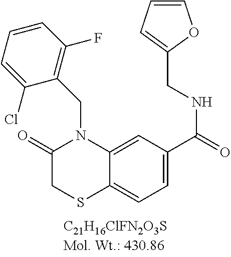 [SMILES: O=C(NCc1ccco1)c1ccc2c(c1)N(Cc1c(F)cccc1Cl)C(=O)CS2]
2′3′-cGAMP, 3′3′-cGAMP, c-di-AMP, c-di-GMP, cAIMP, cAIMP Difluor, cAIM(PS)2, Difluor (Rp/Sp), 2′2″-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2, 2′3′-cGAM(PS)2(Rp/Sp), 2′2′-cGAM(PS)2, 2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2, 2′3′-c-di-AM(PS)2 (Rp,Rp), 2′2′-c-di-AM(PS)2, 3′3′-c-di-AM(PS)2, c-di-AMP Fluorinated, 2′3′-cdAMP Fluorinated, 2′2′-cdAMP Fluorinated, 3′3″-cdAMP Fluorinated, cdGMP, 2′3′-cdGMP, 2′2′-cdGMP, 3′3′-cdGMP, c-di-GM(PS)2, 2′3′-c-di-GM(PS)2, 2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIMP Fluorinated, cAIM(PS)2 Difluor, 3′3′-cAIM(PS)2 Difluor (Rp/Sp), 2′3′-cAIM(PS)2 Difluor, 2′2′-cAIM(PS)2 Difluor, c-di-IMP, 2′3′-cdIMP, 2′2′-cdIMP, 3′3′-cdIMP, c-di-IM(PS)2, 2′3′-c-di-IM(PS)2, 2′2′-c-di-IM(PS)2, 3′3′-c-di-IM(PS)2, c-di-IMP Fluorinated, 2′3′-cdIMP Fluorinated, 2′2′-cdIMP Fluorinated, 3′3′-cdIMP Fluorinated, and amidobenzimidazole (ABZI)-based compounds.

17. The composition of claim 1, wherein the therapeutic agent is an immunotherapy.

18. The composition of claim 17, wherein the immunotherapy is a PD-1 inhibitor such as a PD-1 antibody, a PD-L1 inhibitor such as a PD-L1 antibody, a CTLA-4 inhibitor such as a CTLA-4 antibody, a CSF-1 R inhibitor, an IDO inhibitor, an A1 adenosine inhibitor, an A2A adenosine inhibitor, an A2B adenosine inhibitor, an A3A adenosine inhibitor, an arginase inhibitor, and an HDAC inhibitor.

19. The composition of claim 1, wherein the therapeutic agent is capable of treating, preventing, and/or ameliorating the symptoms of any type of pancreatitis (e.g., ampicillin, imipenem/cilastatin, ceftriaxone sodium).

20. The composition of claim 1, wherein the therapeutic agent is capable of treating, preventing, and/or ameliorating the symptoms of any type of diabetes (e.g., any type of insulin, any type of biguanide (e.g., any type or derivative of metformin), any type of alpha-glucosidase inhibitor (e.g., acarbose, miglitol), any type of dopamine agonist (e.g., bromocriptine), any type of dipeptidyl peptidase-4 inhibitor (e.g., alogliptin (Nesina), alogliptin-metformin (Kazano), alogliptin-pioglitazone (Oseni), linagliptin (Tradjenta), linagliptin-empagliflozin (Glyxambi), linagliptin-metformin (Jentadueto), saxagliptin (Onglyza), saxagliptin-metformin (Kombiglyze XR), sitagliptin (Januvia), sitagliptin-metformin (Janumet and Janumet XR), sitagliptin and simvastatin (Juvisync)), any type of glucagon-like peptide-1 receptor agonist (e.g., albiglutide (Tanzeum), dulaglutide (Trulicity), exenatide (Byetta), exenatide extended-release (Bydureon), liraglutide (Victoza), semaglutide (Ozempic)), any type meglitinide (e.g., nateglinide (Starlix), repaglinide (Prandin), repaglinide-metformin (Prandimet)), any type of sodium-glucose transporter 2 inhibitor (e.g., dapagliflozin (Farxiga), dapagliflozin-metformin (Xigduo XR), canagliflozin (Invokana), canagliflozin-metformin (Invokamet), empagliflozin (Jardiance), empagliflozin-linagliptin (Glyxambi), empagliflozin-metformin (Synjardy), ertugliflozin (Steglatro)), any type of sulfonylurea (e.g., glimepiride (Amaryl), glimepiride-pioglitazone (Duetact), glimepiride-rosiglitazone (Avandaryl), gliclazide, glipizide (Glucotrol), glipizide-metformin (Metaglip), glyburide (DiaBeta, Glynase, Micronase), glyburide-metformin (Glucovance), chlorpropamide (Diabinese), tolazamide (Tolinase), tolbutamide (Orinase, Tol-Tab)), any type of thiazolidinedione (e.g., rosiglitazone (Avandia), rosiglitazone-glimepiride (Avandaryl), rosiglitazone-metformin (Amaryl M), pioglitazone (Actos), pioglitazone-alogliptin (Oseni), pioglitazone-glimepiride (Duetact), and pioglitazone-metformin (Actoplus Met, Actoplus Met XR)).

21. The composition of claim 1,
wherein the therapeutic agent is capable of treating, preventing, and/or ameliorating the symptoms of any type of pancreatic cancer (e.g., Capecitabine (Xeloda), Erlotinib (Tarceva), Fluorouracil (5-FU), Gemcitabine (Gemzar), Irinotecan (Camptosar), Leucovorin (Wellcovorin), Nab-paclitaxel (Abravane), Nanoliposomal irinotecan (Onivyde), Oxaliplatin (Eloxatin)), and/or
wherein the therapeutic agent is capable of treating, preventing, and/or ameliorating the symptoms of any type of cholangitis (e.g., ursodeoxycholic acid, obeticholic acid, fibrates, budesonide).

22. The composition of claim 1, wherein the therapeutic agent is capable of treating, preventing, and/or ameliorating the symptoms of any type of bile duct cancer (e.g., 5-fluorouracil (5-FU), Gemcitabine (Gemzar), Cisplatin (Platinol), Capecitabine (Xeloda), Oxaliplatin (Eloxatin)).

23. The composition of claim 1, wherein the therapeutic agent is capable of treating, preventing, and/or ameliorating the symptoms of any type of gallstone (e.g., ursodiol link (Actigall) and chenodiol link (Chenix)).

24. The composition of claim 1, wherein the therapeutic agent is capable of treating, preventing, and/or ameliorating the symptoms of any type of gall bladder cancer (e.g., 5-fluorouracil (5-FU), Gemcitabine (Gemzar), Cisplatin (Platinol), Capecitabine (Xeloda), Oxaliplatin (Eloxatin)).

25. The composition of claim 1, wherein the therapeutic agent is capable of treating, preventing, and/or ameliorating the symptoms of any type of hepatitis (e.g., any type of antiviral medication (e.g., entecavir (Baraclude), tenofovir (Viread), lamivudine (Epivir), adefovir (Hepsera), telbivudine (Tyzeka)), interferon alfa-2b).

26. The composition of claim 1, wherein the therapeutic agent is capable of treating, preventing, and/or ameliorating the symptoms of any type of liver cancer (e.g., Atezolizumab, Avastin (Bevacizumab), Bevacizumab, Cabometyx (Cabozantinib-S-Malate), Cabozantinib-S-Malate, Cyramza (Ramucirumab), Keytruda (Pembrolizumab), Lenvatinib Mesylate, Lenvima (Lenvatinib Mesylate), Nexavar (Sorafenib Tosylate), Nivolumab, Opdivo (Nivolumab), Pemazyre (Pemigatinib), Pembrolizumab, Pemigatinib, Ramucirumab, Regorafenib, Sorafenib Tosylate, Stivarga (Regorafenib), Tecentriq (Atezolizumab)).

27. The composition of claim 1, wherein the therapeutic agent is any type or kind of chemotherapeutic agent. In some embodiments, the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (TAXOL), pilocarpine, prochloroperazine, rituximab, tamoxifen, taxol, topotecan hydrochloride, trastuzumab, vinblastine, vincristine and vinorelbine tartrate.

28. The composition of claim 1, wherein the therapeutic agent is any type of immunotherapy (e.g., a PD-1 inhibitor such as a PD-1 antibody, a PD-L1 inhibitor such as a PD-L1 antibody, a CTLA-4 inhibitor such as a CTLA-4 antibody, a CSF-1 R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor).

29. The composition of claim 1, wherein the composition or the biodegradable agent further comprises a diagnostic agent.

30. The composition of claim 29, wherein the diagnostic agent is selected from Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase Clostridium histolyticum, Darbepoetin alfa, Denileukin diftitox, Dornase alfa, Dulaglutide, Ecallantide, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1 a, Interferon beta-1 b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a, Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, Ziv-aflibercept, tuberculin purified protein derivative, hyrotropin alpha, secretin, soluble transferrin receptor, troponin, B-type natriuretic peptide, iobenguane I 123, florbetapir F 18, perflutren, gadoterate meglumine, florbetaben F 18, flutemetamol F 18, gadoterate meglumine, isosulfan blue, regadenoson, technetium Tc 99m tilmanocept, florbetaben F 18, perflutren, regadenoson, and flutemetamol F 18.

31. The composition of claim 1, wherein the composition or the biodegradable agent further comprises an agent capable of relaxing (e.g., opening, dilating) the hepatopancreatic anatomical region (e.g., hepatopancreatic ampulla, hepatopancreatic duct, Ampulla of Vater, Sphincter of Oddi) of a human subject.

32. The composition of claim 31, wherein the agent capable of relaxing (e.g., opening, dilating) the hepatopancreatic anatomical region (e.g., hepatopancreatic ampulla, hepatopancreatic duct, Ampulla of Vater, Sphincter of Oddi) is selected from nitroglycerin, scopolamine butylbromide, hymecromone (7-hydroxy-4-methylumbelliferone), botulinum toxin, vasoactive intestinal polypeptide, cholecystokinin-8, and secretin.

33. The composition of claim 1, wherein the one or more biodegradable agents (e.g., microparticles or a nanoparticles) are associated with a therapeutic agent, a diagnostic agent, and an agent capable of relaxing (e.g., opening, dilating) the hepatopancreatic anatomical region.

34. A method for treating, preventing, and/or ameliorating the symptoms of a condition in a subject, the method comprising administering to a subject a pharmaceutically effective amount of a composition as recited in claim 1.

35. The method of claim 34, wherein the composition is co-administered with an agent capable of relaxing (e.g., opening, dilating) the hepatopancreatic anatomical region in the subject (e.g., nitroglycerin, scopolamine butylbromide, hymecromone (7-hydroxy-4-methylumbelliferone), botulinum toxin, vasoactive intestinal polypeptide, cholecystokinin-8, and secretin).

36. The method of claim 35, wherein the agent capable of relaxing (e.g., opening, dilating) the hepatopancreatic anatomical region is administered prior to administration of the composition.

37. The method of claim 35, wherein the agent capable of relaxing (e.g., opening, dilating) the hepatopancreatic anatomical region is administered after administration of the composition.

38. The method of claim 35, wherein the agent capable of relaxing (e.g., opening, dilating) the hepatopancreatic anatomical region is administered concurrently with administration of the composition.

39. The method of claim 34, wherein the subject is a human subject.

40. The method of claim 34, wherein the composition is administered orally (e.g., oral gavage).

41. The method of claim 34, wherein the composition is administered orally, intratumorally, topically, intravenously, or subcutaneously.

42. The method of claim 34, wherein the composition is administered to the pancreas, liver or gallbladder through the hepatopancreatic ducts or/and ampulla of Vater (e.g., pancreatic duct or/and common bile duct).

43. The method of claim 34, wherein the condition is
any disease related to the pancreas (e.g., any type of pancreatic cancer, pancreatitis, diabetes),
any disease related to the bile duct (e.g., cholangitis, bile duct leaks, biliary stricture, biliary stones, bile duct cancer (e.g., cholangiocarcinoma),
any disease related to the gall bladder (e.g., gall stones, gall bladder carcinoma), and any disease related to the liver (e.g., hepatitis, cholangitis, biliary atresia, alpha-1 antitrypsin deficiency, alagille syndrome, progressive familial intrahepatic cholestasis, Langerhans cell histiocytosis, hepatic hemangioma, polycystic liver disease, liver cancer).

44. A kit comprising a comprising
a) a composition described in claim 1; and
b) one or more of
therapeutic agents, and
an agent capable of relaxing (e.g., opening, dilating) the hepatopancreatic anatomical region in the subject (e.g., nitroglycerin, scopolamine butylbromide, hymecromone (7-hydroxy-4-methylumbelliferone), botulinum toxin, vasoactive intestinal polypeptide, cholecystokinin-8, and secretin).
